SEQUENCE LISTING (18 sequences)

SEQ ID NO: 1 - DNA, 57 bp, Artificial Sequence
  Synthetic Primer
  misc_feature (22)..(37): n is a, c, g, or t
cgacgtaaaacgacggccagtnnnnnnnnnnnnnnnnggtgaaaccccgtctctaca

SEQ ID NO: 2 - DNA, 56 bp, Artificial Sequence
  Synthetic Primer
  misc_feature (22)..(37): n is a, c, g, or t
cgacgtaaaacgacggccagtnnnnnnnnnnnnnnnnggtgaaaccccgtctctac

SEQ ID NO: 3 - DNA, 57 bp, Artificial Sequence
  Synthetic Primer
  misc_feature (22)..(37): n is a, c, g, or t
cgacgtaaaacgacggccagtnnnnnnnnnnnnnnnnggtgaaaccccgtctctact

SEQ ID NO: 4 - DNA, 59 bp, Artificial Sequence
  Synthetic Primer
  misc_feature (22)..(37): n is a, c, g, or t
cgacgtaaaacgacggccagtnnnnnnnnnnnnnnnncatgcctgtagtcccagctact

SEQ ID NO: 5 - DNA, 62 bp, Artificial Sequence
  Synthetic Primer
  misc_feature (22)..(37): n is a, c, g, or t
cgacgtaaaacgacggccagtnnnnnnnnnnnnnnnnatagtgaaaccccatctctacaaaa

SEQ ID NO: 6 - DNA, 58 bp, Artificial Sequence
  Synthetic Primer
  misc_feature (22)..(37): n is a, c, g, or t
cgacgtaaaacgacggccagtnnnnnnnnnnnnnnnnggtgaaaccccatctctacaa

SEQ ID NO: 7 - DNA, 61 bp, Artificial Sequence
  Synthetic Primer
  misc_feature (22)..(37): n is a, c, g, or t
cgacgtaaaacgacggccagtnnnnnnnnnnnnnnnnatagtgaaaccccatctctacaaa

SEQ ID NO: 8 - DNA, 55 bp, Artificial Sequence
  Synthetic Primer
  misc_feature (22)..(37): n is a, c, g, or t
cgacgtaaaacgacggccagtnnnnnnnnnnnnnnnngaggtgggaggattgctt

SEQ ID NO: 9 - DNA, 55 bp, Artificial Sequence
  Synthetic Primer
  misc_feature (22)..(37): n is a, c, g, or t
cgacgtaaaacgacggccagtnnnnnnnnnnnnnnnnaccagcctgggcaacata

SEQ ID NO: 10 - DNA, 49 bp, Artificial Sequence
  Synthetic Primer
cacacaggaaacagctatgaccatgcctcctaagtagctgggactacag

SEQ ID NO: 11 - DNA, 49 bp, Artificial Sequence; identical to SEQ ID NO: 10
  Synthetic Primer

SEQ ID NO: 12 - DNA, 49 bp, Artificial Sequence; identical to SEQ ID NO: 10
  Synthetic Construct

SEQ ID NO: 13 - DNA, 60 bp, Artificial Sequence
  Synthetic Primer
cacacaggaaacagctatgaccatgtgcagtggcacgatcatagctcactgcagccttga

SEQ ID NO: 14 - DNA, 44 bp, Artificial Sequence
  Synthetic Construct
cacacaggaaacagctatgaccatgctcccgagtagctgggact

SEQ ID NO: 15 - DNA, 46 bp, Artificial Sequence
  Synthetic Primer
cacacaggaaacagctatgaccatgctcccgagtagctgggactac

SEQ ID NO: 16 - DNA, 45 bp, Artificial Sequence
  Synthetic Primer
cacacaggaaacagctatgaccatgcccgagtagctgggactaca

SEQ ID NO: 17 - DNA, 42 bp, Artificial Sequence
  Synthetic Primer
cacacaggaaacagctatgaccatgaggctggagtgcagtgg

SEQ ID NO: 18 - DNA, 42 bp, Artificial Sequence
  Synthetic Primer
cacacaggaaacagctatgaccatgccaccatgcctggctaa

(19) United States
(12) Patent Application Publication
(10) Pub. No.: US 2024/0068033 A1
(43) Pub. Date: Feb. 29, 2024

(54) METHODS OF DETECTING HIGH RISK BARRETT'S ESOPHAGUS WITH DYSPLASIA, AND ESOPHAGEAL ADENOCARCINOMA

(71) Applicants: CASE WESTERN RESERVE UNIVERSITY, Cleveland, OH (US); THE JOHNS HOPKINS UNIVERSITY, Baltimore, MD (US)

(72) Inventors: Sanford Markowitz, Pepper Pike, OH (US); Amitabh Chak, University Heights, OH (US); Helen Moinova, Cleveland, OH (US); Joseph Willis, Shaker Heights, OH (US); Bert Vogelstein, Baltimore, MD (US); Kenneth Kinzler, Baltimore, MD (US); Nickolas Papadopoulos, Baltimore, MD (US); Chetan Bettewgowda, Baltimore, MD (US); Christopher Douville, Baltimore, MD (US)

(21) Appl. No.: 18/261,366

(22) PCT Filed: Jan. 14, 2022
(86) PCT No.: PCT/US2022/012423

§ 371 Date: Jul. 13, 2023

Related U.S. Application Data
(60) Provisional application No. 63/137,546, filed on Jan. 14, 2021.

(51) Int. Cl. C12Q 1/6883

(52) U.S. Cl. CPC C12Q 1/6883; C12Q 2600/118; C12Q 2600/156

(57) ABSTRACT

A method of detecting Barrett's esophagus with low grade dysplasia, or Barrett's esophagus with high grade dysplasia, or adenocarcinoma of the esophagus, applying a Repetitive Element Aneuploidy Sequencing System (RealSeqS) methodology to a biological sample from the esophagus of the subject to detect Barrett's esophagus with low grade dysplasia, or Barrett's esophagus with high grade dysplasia, or adenocarcinoma of the esophagus.

### RELATED APPLICATION

This application claims priority from U.S. Provisional Application No. 63/137,546, filed Jan. 14, 2021, the subject matter of which is incorporated herein by reference in its entirety.

### GOVERNMENT FUNDING

This invention was made with government support under Grant No. CA152756, CA150964, CA163060 awarded by The National Institutes of Health. The United States government has certain rights in the invention.

### SEQUENCE LISTING

The instant application contains a Sequence Listing which has been submitted in ASCII format via EFS-Web and is hereby incorporated by reference in its entirety. Said ASCII copy, created on Jan. 13, 2022, is named CWR030249WOORDSEQUENCELISTING.txt and is 4,567 bytes in size.

### BACKGROUND

Esophageal adenocarcinoma (EAC) is a rapidly rising, refractory cancer, with an overall five-year survival that remains below 20%. Barrett's esophagus (BE), a condition of intestinal metaplasia of the distal esophagus associated with chronic gastroesophageal reflux disease (GERD) and other risk factors, is the only known precursor to EAC. BE progresses stepwise from metaplasia (non-dysplastic BE, NDBE), to low grade dysplasia (LGD), then to high grade dysplasia (HGD), and finally to carcinoma. Although most BE cases do not progress, those that do progress to cancer likely do so through acquired genetic and epigenetic alterations. Current strategies for prevention and early detection of EAC are based on endoscopic detection of BE, with subsequent surveillance to identify dysplasia on random biopsies obtained during endoscopy, followed by endoscopic ablation of the dysplastic tissue. While surveillance and ablation of dysplasia is effective in reducing the risk of developing invasive cancers, sampling with random biopsies is inherently imprecise. In addition, there remains a well-recognized risk of development of interval cancers between surveillance examinations. Perhaps most importantly, sole reliance on histopathologic criteria for recognition of dysplastic BE is imperfect, as reflected by poor inter-observer agreement even among expert pathologists. Moreover, the related inability to identify the majority of patients with BE who are at low risk for progression to cancer leads to over-surveillance of the total population of BE patients.

Aneuploidy and tetraploidy have long been recognized to accompany progression from non-dysplastic BE to dysplasia and early EAC and have been proposed to be predictive biomarkers for identifying BE at high risk of such progression. Methods for detecting aneuploidy have traditionally employed biopsies that capture only focal regions of the affected esophageal segment, and that therefore must be repeated across multiple locations to obtain, at best, a somewhat representative sampling. In part for this reason, aneuploidy has not been incorporated into routine clinical practice. Additionally, the relationship between aneuploidy, the risk of progression, and actual progression has not yet been defined. Other approaches to assess the risk of progression in patients with BE include in vitro imaging and flow cytometry of biopsied material. These approaches have also not been widely used, either because they are technically cumbersome, are low throughput, or have special requirements.

### SUMMARY

Embodiments described herein relate to a system and methods of detecting Barrett's esophagus (BE) with low grade dysplasia (LGD), or Barrett's esophagus (BE) with high grade dysplasia (HGD), or adenocarcinoma of the esophagus (EAC); methods of detecting or determining a subject with BE at increased risk of progression to LGD, or HGD, or EAC; and/or methods of detecting a subject with BE with LGD at increased risk of progression to HGD, or EAC by identifying and/or detecting one or more chromosomal anomalies (e.g., aneuploidies) in a biological sample, such as brushing, from the esophagus of the subject.

It was found that the combination of esophageal samples (e.g., brushings) combined with a streamlined massively parallel sequencing approach, such as RealSeqS analysis, provides a practical and sensitive method for detecting chromosome arm alterations in BE patients and can be used to discriminate different stages of BE progression and detect the majority of prevalent histologically advanced lesions.

Esophageal brushings can sample a much more extensive region of the esophagus than conventional biopsies, even when multiple biopsies are performed. But this extensive and convenient sampling comes with a cost: the aneuploidy present in the dysplastic cells within any individual lesion is diluted with non-dysplastic cells from the remaining esophagus. Advantageously, the aneuploidy detection methods described herein can be used for the detection of a relatively small fraction of aneuploid cells admixed with a much larger number of non-aneuploid cells.

Aneuploidy typifies EAC and HGD, but also can be identified in a small proportion of non-dysplastic BE (NDBE) patients and a larger proportion of LGD patients. Alterations of specific chromosome arms containing well-known driver genes are found commonly in late stage but rarely in early stage disease. These chromosomal alterations can be used to design a molecular classifier that can accurately discriminate patients with NDBE from those with progression to dysplasia or cancer, and identifies a subset of NDBE patients with a molecular signature of progression.

In some embodiments, the one or more chromosomal anomalies (e.g., aneuploidies) in the biological sample from the esophagus of the subject can be detected using a Repetitive Element Aneuploidy Sequencing System (RealSeqS) methodology that identifies both global aneuploidy and individual chromosome alterations in the biological sample. Global aneuploidy identified by application of the RealSeqS methodology to the biological sample can be used to determine a global aneuploidy score (GAS) that can be used to determine aneuploidy of the biological sample and distinguish subjects with non-dysplastic Barrett's esophagus (NDBE) from those with BE with LGD, BE with HGD, and AEC. When combined with data on individual chromosome alterations in the biological sample generated using the RealSeqS methodology, the combined GAS score and the identified individual chromosome alterations can be used to provide a Barrett's Aneuploidy Decision (BAD) classifier for distinguishing stages of BE progression. The BAD classifier can be used to determine treatment of the subject. Subjects identified as having an intermediate risk or high risk classification of progression of Barrett's esophagus can be treated, for example, with an endoscopic ablation therapy, endoscopic photodynamic therapy, endoscopic cryotherapy, endoscopic mucosal resection, a surgical resection therapy, a non-endoscopic surgical therapy, or systemic therapy. The subjects identified as having a low risk of progression can have surveillance with reduced frequency.

In some embodiments, the method can include applying a RealSeqS methodology to a biological sample from the esophagus of the subject to detect BE with LGD, or BE with HGD, or EAC.

In other embodiments, the method can include applying a RealSeqS methodology to a biological sample from the esophagus of the subject to detect BE at increased risk of progression to LGD, or HGD, or EAC.

In still other embodiments, the method can include applying a RealSeqS methodology to a biological sample from the esophagus of the subject to detect Barrett's esophagus with low grade dysplasia at increased risk of progression to high grade dysplasia, or adenocarcinoma.

This disclosure also provides methods and materials for detecting and treating BE with LGD, or BE with HGD, or EAC; methods of detecting and treating a subject with BE at increased risk of progression to LGD, or HGD, or EAC; and/or methods of detecting and treating a subject with BE with LGD at increased risk of progression to HGD, or EAC. In some cases, one or more chromosomal anomalies can be identified in DNA (e.g., genomic DNA) obtained from a biological sample from the esophagus of the subject. For example, a mammal (e.g., human) can be identified as having LGD, HGD, or EAC, at least in part, on the presence of one or more chromosomal anomalies. In some embodiments, a mammal identified as having a LGD, HGD, or EAC based, at least in part, on one or more chromosomal abnormalities can be assessed for the purposes of treating BE. In some embodiments, a mammal identified as having LGD, HGD, or EAC based, at least in part, on the presence of one or more chromosomal anomalies can be treated with one or more cancer treatments.

In some embodiments, the application of RealSeqS methodology comprises determining a global aneuploidy score (GAS).

In other embodiments, the RealSeqS methodology includes (i) amplifying unique loci of genomic nucleic acid of the sample, (ii) matching the unique loci to a control, (iii) calculating the statistical gains or losses for each of non-acrocentric chromosome arms, (iv) integrating the chromosome arms into a global aneuploidy score (GAS) using machine learning, and (v) quantifying chromosome arm levels and querying focal changes of interest.

Other embodiments relate to a method of detecting Barrett's esophagus with low grade dysplasia, or Barrett's esophagus with high grade dysplasia, or adenocarcinoma of the esophagus. The method includes applying a Repetitive Element Aneuploidy Sequencing System (RealSeqS) methodology to a biological sample from the esophagus of a subject to determine the global aneuploidy score (GAS) and/or to identify copy number alterations in a panel of chromosome alterations, the chromosomal alterations including chromosome gains of any of chromosome regions 8q24, 1q, 7p, 20q, 2q, 13q, 5p, 12p and/or losses of any of chromosome regions 5q, 17p, 4p, 4q, 9p, 18q, 16q, 21q, 22q, 10p.

Still other embodiments relate to a method of detecting a subject with Barrett's esophagus at increased risk of progression to low grade dysplasia, or high grade dysplasia, or adenocarcinoma. The method includes applying a Repetitive Element Aneuploidy Sequencing System (RealSeqS) methodology to a biological sample from the esophagus of the subject to determine the global aneuploidy score and/or to identify copy number alterations in a panel of chromosome alterations, the chromosome alterations including: chromosome gains of any of chromosome regions 8q24, 1q, 7p, 20q, 2q, 13q, 5p, 12p and/or losses of any of chromosome regions 5q, 17p, 4p, 4q, 9p, 18q, 16q, 21q, 22q, 10p.

Further embodiments relate to a method of detecting a subject with Barrett's esophagus with low grade dysplasia at increased risk of progression to high grade dysplasia, or adenocarcinoma. The method includes applying a Repetitive Element Aneuploidy Sequencing System (RealSeqS) methodology to a biological sample from the esophagus of the subject to determine the global aneuploidy score and/or to identify copy number alterations in a panel of chromosome alterations, the chromosome alterations including: chromosome gains of any of chromosome regions 8q24, 1q, 7p, 20q, 2q, 13q, 5p, 12p and/or losses of any of chromosome regions 5q, 17p, 4p, 4q, 9p, 18q, 16q, 21q, 22q, 10p.

In some embodiments, the global aneuploidy score indicative of the presence of dysplasia or cancer, or increased risk of progression to low grade dysplasia, or high grade dysplasia, or cancer, is >0.4, or >0.6, or >0.8, or >0.9, or >0.907.

In other embodiments, copy number alterations are determined in a panel of chromosome alterations comprising: chromosome gains of any of chromosome regions 8q24, 1q, 20q, 12p and/or losses of any of chromosome regions 17p, 9p, 10p.

In other embodiments, the global aneuploidy score indicative of presence of dysplasia or cancer, or increased risk of progression to low grade dysplasia, or high grade dysplasia, or cancer, is >0.4, or >0.6, or >0.8, or >0.9, or >0.907 and copy number alterations are determined in a panel of chromosome alterations, the chromosome alterations including chromosome gains of any of chromosome regions 8q24, 1q, 20q, 12p and/or losses of any of chromosome regions 17p, 9p, 10p.

In some embodiments, the global aneuploidy score indicative of presence of dysplasia or cancer, or increased risk of progression to low grade dysplasia, or high grade dysplasia, or cancer, is >0.6, and copy number alterations are determined in a panel of chromosome alterations comprising: chromosome gains of any of chromosome regions 8q24, 1q, 20q, 12p and/or losses of any of chromosome regions 17p, 9p, 10p.

In other embodiments, the global aneuploidy score indicative of presence of dysplasia or cancer, or increased risk of progression to low grade dysplasia, or high grade dysplasia, or cancer, is >0.6, and copy number alterations are determined in a panel of chromosome alterations comprising: chromosome gains of any of chromosome regions 8q24, 1q, 20q, 12p and/or losses of any of chromosome regions 17p, 9p.

In some embodiments, chromosomal gains are identified by any of values of Z_{w} (also denoted as Z) of >2.0, >2.1>, >2.2> <2.3, >2.4>, >2.5, >2.6, >2.7, >2.8, >2.9, >3.0, or by a value exceeding a cutoff between 2.0 to 3.0, and chromosomal losses are identified by any of values of Z_{w} (also denoted as Z) of <−2.0, <−2.1, <−2.2, <−2.3, <−2.4, <−2.5, <−2.6, <−2.7, <−2.8, <−2.9, <−3.0, or by a value lower than a cutoff between −2.0 to −3.0.

In some embodiments, methods of detecting BE with LGD, or BE with HGD, or EAC; methods of detecting a subject with BE at increased risk of progression to LGD, or HGD, or EAC; and/or methods of detecting a subject with BE with LGD at increased risk of progression to HGD, or EAC can include detecting and/or identifying chromosomal anomalies in the biological sample of the esophagus obtained from subject. The biological sample can include a brushing, scraping, biopsy, or surgical resection of cells from the subject. The sample may be collected via random endoscopic sampling, computer-assisted endoscopic sampling, image-guided endoscopic sampling, or non-endoscopic sampling via brushing, abrasion or scraping. In some embodiments, the sample can be an esophageal brushing that includes a mixture of normal epithelium, non-dysplastic Barrett's epithelium, and dysplastic epithelium.

In some embodiments, the biological sample of the esophagus is a brushing sample.

In some embodiments, the brushing sample is obtained by a cytology brush.

In other embodiments, the brushing sample is obtained by a balloon sampling device.

In other embodiments, the brushing sample is frozen.

In some embodiments, if the subject is determined to have BE with LGD, BE with HGD, or EAC, then the method further comprises administering to the subject cryotherapy, photodynamic therapy (PDT); radiofrequency ablation (RFA); laser ablation; argon plasma coagulation (APC); electrocoagulation (electrofulguration); esophageal stent, surgery, and/or a therapeutic agent.

In some embodiments, the therapeutic agent is a proton pump inhibitor, a Histamine H2 receptor blocking agents, an anti-reflux medication, a drug that moves food thru the gastrointestinal tract more quickly, carboplatin and paclitaxel (Taxol), which is optionally administered in combination with radiation; cisplatin and 5-fluorouracil (5-FU), which optionally administered in combination with radiation; ECF: epirubicine (Ellence), cisplatin, and 5-FU; DCF: docetaxel (Taxotere), cisplatin, and 5-FU; Cisplatin with capecitabine (Xeloda); oxaliplatin and either 5-FU or capecitabine; doxorubicin (Adriamycin), bleomycin, mitomycin, methotrexate, vinorelbine (Navelbine), topotecan, and irinotecan (Camptosar), trastuzumab, and/or ramucirumab.

In some embodiments, the surgery is endoscopic mucosal resection (EMR), esophagectomy, and/or anti-reflux surgery.

Other embodiments described herein relate to method of treating a subject having Barrett's esophagus with low grade dysplasia, or Barrett's esophagus with high grade dysplasia, or adenocarcinoma of the esophagus, wherein it has been previously determined that a sample from the esophagus of the subject Barrett's esophagus with low grade dysplasia, or Barrett's esophagus with high grade dysplasia, or adenocarcinoma of the esophagus has a GAS of >0.1, >0.2, >0.3, >0.4, or >0.6, or >0.8, or >0.9, or >0.907 and at least one copy number alteration in a panel of chromosome alterations, the chromosome alterations including: chromosome gains of any of chromosome regions 8q24, 1q, 7p, 20q, 2q, 13q, 5p, 12p and/or losses of any of chromosome regions 5q, 17p, 4p, 4q, 9p, 18q, 16q, 21q, 22q, 10p. The method includes administering to the subject cryotherapy, photodynamic therapy (PDT); radiofrequency ablation (RFA); laser ablation; argon plasma coagulation (APC); electrocoagulation (electrofulguration); esophageal stent, surgery, and/or a therapeutic agent.

### BRIEF DESCRIPTION OF THE DRAWINGS

FIGS. 1(A-E) illustrate the overview of the Repetitive Element Aneuploidy Sequencing System (RealSeqS) methodology approach. A) A single primer pair concomitantly amplifies ˜350,000 unique loci spread throughout the genome. B) The patient sample is matched to the 7 closest control samples. C) The statistical significance of gains and losses for each of the 39 non-acrocentric chromosome arms is calculated. D) The 39 chromosome arms are integrated into a Genome Aneuploid Score (GAS) using a supervised machine learning model. E) Chromosome arm levels can be quantified and focal changes of interest queried.

FIGS. 2(A-D) illustrate the performance of the Genome Aneuploid Score (GAS) to discriminate samples from patients with HGD or EAC from samples from individuals with NDBE. (A) The Receiver Operating Characteristic (ROC) curve and area under the curve (AUC) for the GAS metric as applied to the Training Set. (B) Violin plot of the GAS distribution among the clinical subsets of the Training Set. Individuals with LGD were excluded from the Training Set. (C) ROC curve and AUC for the GAS metric as applied to the Validation Set. (D) Violin plot of the GAS distribution among the clinical subsets of the Validation Set.

FIGS. 3(A-C) illustrate the BAD Molecular Classification of progression to dysplasia in patients with Barrett's Esophagus. A) The BAD Decision Tree Algorithm. B) Heatmap of predictive features used in the BAD classifier depicted for Training Set samples that do or do not meet criteria as Very-BAD. C) Heatmap of predictive features used in the BAD classifier depicted for Validation Set samples that do or do not meet criteria as Very-BAD.

FIG. 4 is a schematic of the progression of aneuploidy, chromosomal arm alterations, and the BAD classification going successively from Non-dysplastic Barrett's Esophagus to Adenocarcinoma. Chromosome alterations shown in red contribute to the BAD classifier algorithm.

FIG. 5 illustrates plots showing representative examples of gains of chromosome 8q and of focal gains of 8q24.

FIG. 6 illustrates a plot showing the length of BE segment in all of the NDBE patients from the combined Training and Validation Sets compared according to their BAD classification. The boxes are drawn to include the 1^{st} through 3^{rd} quartile range, with median indicated by the horizontal line within the box. The ends of the whiskers represent one and a half times the interquartile range (1.5*IQR). The P-values of between-group comparisons were calculated using Welsh test for independent samples with unequal group variances. Overall P=0.7878 for the F-test by one-way ANOVA.

FIGS. 7(A-C) illustrate plots showing the characterization of Aneuploidy. (A) Violin plots for the number of altered chromosome arms (Z>2.5 or Z<−2.5) in those patients whose samples had GAS>0.6. (B) Fraction of samples with representative chromosome arm gains and losses (Z>2.5 or Z<−2.5) in Training Set samples with GAS>0.6. (C) Fraction of samples with representative chromosome arm gains and losses in Validation Set samples with GAS>0.6. Graphed Bars denote group means and error bars represent 95% CIs.

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this invention belongs.

Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Each embodiment of the invention described herein may be taken alone or in combination with one or more other embodiments of the invention.

Throughout this specification, the word “comprise” or variations, such as “comprises” or “comprising” will be understood to imply the inclusion of a stated integer or groups of integers but not the exclusion of any other integer or group of integers.

The articles “a” and “an” are used herein to refer to one or to more than one (i.e., to at least one) of the grammatical object of the article. By way of example, “an element” means one element or more than one element.

The terms “adenoma” is used herein to describe any precancerous neoplasia or benign tumor of epithelial tissue, for example, a precancerous neoplasia of the gastrointestinal tract, pancreas, and/or the bladder.

The term “esophagus” is intended to encompass the upper portion of the digestive system spanning from the back of the oral cavity, passing downwards through the rear part of the mediastinum, through the diaphragm and into the stomach.

The term “esophageal cancer” is used herein to refer to any cancerous neoplasia of the esophagus.

“Barrett's esophagus” as used herein refers to an abnormal change (metaplasia) in the cells of the lower portion of the esophagus. Barrett's is characterized the finding of intestinal metaplasia in the esophagus.

A “brushing” of the esophagus, as referred to herein, may be obtained using any of the means known in the art. In some embodiments, a brushing is obtained by contacting the esophagus with a brush, a cytology brush, a sponge, a balloon, or with any other device or substance that contacts the esophagus and obtains an esophageal sample.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “detection” is used herein to refer to any process of observing a marker, or a change in a marker, in a biological sample, whether or not the marker or the change in the marker is actually detected. In other words, the act of probing a sample for a marker or a change in the marker, is a “detection” even if the marker is determined to be not present or below the level of sensitivity. Detection may be a quantitative, semi-quantitative or non-quantitative observation.

The term “neoplasia” as used herein refers to an abnormal growth of tissue. As used herein, the term “neoplasia” may be used to refer to cancerous and non-cancerous tumors, as well as to Barrett's esophagus (which may also be referred to herein as a metaplasia) and Barrett's esophagus with dysplasia. In some embodiments, the Barrett's esophagus with dysplasia is Barrett's esophagus with high grade dysplasia. In some embodiments, the Barrett's esophagus with dysplasia is Barrett's esophagus with low grade dysplasia. In some embodiments, the neoplasia is a cancer (e.g., esophageal adenocarcinoma).

“Gastrointestinal neoplasia” refers to neoplasia of the upper and lower gastrointestinal tract. As commonly understood in the art, the upper gastrointestinal tract includes the esophagus, stomach, and duodenum; the lower gastrointestinal tract includes the remainder of the small intestine and all of the large intestine.

As used herein, the term “risk of progression” means the probability of progressing to low grade dysplasia, high grade dysplasia, or esophageal adenocarcinoma.

The terms “healthy”, “normal,” and “non-neoplastic” are used interchangeably herein to refer to a subject or particular cell or tissue that is devoid (at least to the limit of detection) of a disease condition, such as a neoplasia.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position: when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated or “non-homologous” shares, in some embodiments, less than 40% identity, and in particular embodiments, less than 25% identity with a sequence of the present invention. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid and protein sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, word length=12 to obtain nucleotide sequences homologous to nucleic acid molecules of the invention. BLAST protein searches can be performed with the XBLAST program, score=50, word length=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press. New York, 1988; Biocomputing: Informatics and Genome Projects, Smith, D. W., ed., Academic Press, New York, 1993: Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M, and Devereux, J., eds., M Stockton Press, New York, 1991: and Carillo, H., and Lipman, D., SIAM J. Applied Math., 48: 1073, 1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894: Altschul, S., et al., J. Mol. Biol. 215: 403-410 (1990)). The well-known Smith Waterman algorithm may also be used to determine identity.

The term “including” is used herein to mean, and is used interchangeably with, the phrase “including but not limited to.”

The term “isolated” as used herein with respect to nucleic acids, such as DNA or RNA, refers to molecules in a form which does not occur in nature. Moreover, an “isolated nucleic acid” is meant to include nucleic acid fragments which are not naturally occurring as fragments and would not be found in the natural state.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

“Operably linked” when describing the relationship between two DNA regions simply means that they are functionally related to each other. For example, a promoter or other transcriptional regulatory sequence is operably linked to a coding sequence if it controls the transcription of the coding sequence.

The term “or” is used herein to mean, and is used interchangeably with, the term “and/or”, unless context clearly indicates otherwise.

The terms “proteins” and “polypeptides” are used interchangeably herein.

A “sample” includes any material that is obtained or prepared for detection of a molecular marker or a change in a molecular marker, or any material that is contacted with a detection reagent or detection device for the purpose of detecting a molecular marker or a change in the molecular marker.

As used herein “obtaining a sample” includes directly retrieving a sample from a subject to be assayed, or directly retrieving a sample from a subject to be stored and assayed at a later time. Alternatively, a sample may be obtained via a second party. That is, a sample may be obtained via, e.g., shipment, from another individual who has retrieved the sample, or otherwise obtained the sample.

A “subject” is any organism of interest, generally a mammalian subject, such as a mouse, and in particular embodiments, a human subject.

Embodiments described herein relate to a system and methods of detecting Barrett's esophagus (BE) with low grade dysplasia (LGD), or Barrett's esophagus (BE) with high grade dysplasia (HGD), or adenocarcinoma of the esophagus (EAC); methods of detecting or determining a subject with BE at increased risk of progression to LGD, or HGD, or EAC; and/or methods of detecting a subject with BE with LGD at increased risk of progression to HGD, or EAC by identifying and/or detecting one or more chromosomal anomalies (e.g., aneuploidies) in a biological sample, such as brushing, from the esophagus of the subject.

It was found that the combination of esophageal samples (e.g., brushings) combined with a streamlined massively parallel sequencing approach, such as RealSeqS analysis, provides a practical and sensitive method for detecting chromosome arm alterations in BE patients and can be used to discriminate different stages of BE progression and detect the majority of prevalent histologically advanced lesions.

Esophageal brushings can sample a much more extensive region of the esophagus than conventional biopsies, even when multiple biopsies are performed. But this extensive and convenient sampling comes with a cost: the aneuploidy present in the dysplastic cells within any individual lesion is diluted with non-dysplastic cells from the remaining esophagus. Advantageously, the aneuploidy detection methods described herein can be used for the detection of a relatively small fraction of aneuploid cells admixed with a much larger number of non-aneuploid cells.

Aneuploidy typifies EAC and HGD, but also can be identified in a small proportion of non-dysplastic BE (NDBE) patients and a larger proportion of LGD patients. Alterations of specific chromosome arms containing well-known driver genes are found commonly in late stage but rarely in early stage disease. These chromosomal alterations can be used to design a molecular classifier that can accurately discriminate patients with NDBE from those with progression to dysplasia or cancer, and identifies a subset of NDBE patients with a molecular signature of progression.

In some embodiments, methods of detecting BE with LGD, or BE with HGD, or EAC; methods of detecting a subject with BE at increased risk of progression to LGD, or HGD, or EAC; and/or methods of detecting a subject with BE with LGD at increased risk of progression to HGD, or EAC can include detecting and/or identifying chromosomal anomalies in the biological sample of the esophagus obtained from subject. The biological sample can include a brushing, scraping, biopsy, or surgical resection of cells from the subject. The sample may be collected via random endoscopic sampling, computer-assisted endoscopic sampling, image-guided endoscopic sampling, or non-endoscopic sampling via brushing, abrasion or scraping. In some embodiments, the sample can be an esophageal brushing that includes a mixture of normal epithelium, non-dysplastic Barrett's epithelium, and dysplastic epithelium.

Examples of chromosomal anomalies that can be detected using methods and materials described herein include, without limitation, numerical disorders, structural abnormalities, allelic imbalances, and microsatellite instabilities. A chromosomal anomaly can include a numerical disorder. For example, a chromosomal anomaly can include an aneuploidy (e.g., an abnormal number of chromosomes). In some cases, an aneuploidy can include an entire chromosome. In some cases, an aneuploidy can include part of a chromosome (e.g., a chromosome arm gain or a chromosome arm loss). Examples of aneuploidies include, without limitation, monosomy, trisomy, tetrasomy, and pentasomy. A chromosomal anomaly can include a structural abnormality. Examples of structural abnormalities include, without limitation, deletions, duplications, translocations (e.g., reciprocal translocations and Robertsonian translocations), inversions, insertions, rings, and isochromosomes. Chromosomal anomalies can occur on any chromosome pair (e.g., chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21, chromosome 22, and/or one of the sex chromosomes (e.g., an X chromosome or a Y chromosome). For example, aneuploidy can occur, without limitation, in chromosome 13 (e.g., trisomy 13), chromosome 16 (e.g., trisomy 16), chromosome 18 (e.g., trisomy 18), chromosome 21 (e.g., trisomy 21), and/or the sex chromosomes (e.g., X chromosome monosomy; sex chromosome trisomy such as XXX, XXY, and XYY; sex chromosome tetrasomy such as XXXX and XXYY; and sex chromosome pentasomy such as XXXXX, XXXXY, and XYYYY). For example, structural abnormalities can occur, without limitation, in chromosome 4 (e.g., partial deletion of the short arm of chromosome 4), chromosome 11 (e.g., a terminal 11q deletion), chromosome 13 (e.g., Robertsonian translocation at chromosome 13), chromosome 14 (e.g., Robertsonian translocation at chromosome 14), chromosome 15 (e.g., Robertsonian translocation at chromosome 15), chromosome 17 (e.g., duplication of the gene encoding peripheral myelin protein 22), chromosome 21 (e.g., Robertsonian translocation at chromosome 21), and chromosome 22 (e.g., Robertsonian translocation at chromosome 22).

In some embodiments, the one or more chromosomal anomalies (e.g., aneuploidies) in the biological sample from the esophagus of the subject can be detected using a Repetitive Element Aneuploidy Sequencing System (RealSeqS) methodology that identifies both global aneuploidy and individual chromosome alterations in the biological sample. Global aneuploidy identified by application of the RealSeqS methodology to the biological sample can be used to determine a global aneuploidy score (GAS) that can be used to determine aneuploidy of the biological sample and distinguish subjects with non-dysplastic Barrett's esophagus (NDBE) from those with BE with LGD, BE with HGD, and AEC. When combined with data on individual chromosome alterations in the biological sample generated using the RealSeqS methodology, the combined GAS score and the identified individual chromosome alterations can be used to provide a Barrett's Aneuploidy Decision (BAD) classifier for distinguishing stages of BE progression. The BAD classifier can be used to determine treatment of the subject. Subjects identified as having an intermediate risk or high risk classification of progression of Barrett's esophagus can be treated, for example, with an endoscopic ablation therapy, endoscopic photodynamic therapy, endoscopic cryotherapy, endoscopic mucosal resection, a surgical resection therapy, a non-endoscopic surgical therapy, or systemic therapy. The subjects identified as having a low risk of progression can have surveillance with reduced frequency.

In some embodiments, the method can include applying a RealSeqS methodology to a biological sample from the esophagus of the subject to detect BE with LGD, or BE with HGD, or EAC.

In other embodiments, the method can include applying a RealSeqS methodology to a biological sample from the esophagus of the subject to detect BE at increased risk of progression to LGD, or HGD, or EAC.

In still other embodiments, the method can include applying a RealSeqS methodology to a biological sample from the esophagus of the subject to detect Barrett's esophagus with low grade dysplasia at increased risk of progression to high grade dysplasia, or adenocarcinoma.

This disclosure also provides methods and materials for detecting and treating BE with LGD, or BE with HGD, or EAC; methods of detecting and treating a subject with BE at increased risk of progression to LGD, or HGD, or EAC; and/or methods of detecting and treating a subject with BE with LGD at increased risk of progression to HGD, or EAC. In some cases, one or more chromosomal anomalies can be identified in DNA (e.g., genomic DNA) obtained from a biological sample from the esophagus of the subject. For example, a mammal (e.g., human) can be identified as having LGD, HGD, or EAC, at least in part, on the presence of one or more chromosomal anomalies. In some embodiments, a mammal identified as having a LGD, HGD, or EAC based, at least in part, on one or more chromosomal abnormalities can be assessed for the purposes of treating BE. In some embodiments, a mammal identified as having LGD, HGD, or EAC based, at least in part, on the presence of one or more chromosomal anomalies can be treated with one or more cancer treatments.

In the methods, a biological sample from an esophagus of a subject having or suspected of having BE can be obtained. The biological sample from the esophagus can include genomic DNA. The sample can include a brushing, scraping, biopsy, or surgical resection of cells from the subject. The sample may be collected via random endoscopic sampling, computer-assisted endoscopic sampling, image-guided endoscopic sampling, or non-endoscopic sampling via brushing, abrasion or scraping. The brushing sample can be obtained, for example, by a cytology brush or by a balloon sampling device. The sample may be at room temperature or frozen. The sample may be freshly obtained, formalin fixed, alcohol fixed, or paraffin embedded. In some cases, a sample can include an esophageal brushing from a subject that includes a mixture of normal epithelium, non-dysplastic Barrett's epithelium, and dysplastic epithelium.

In some embodiments, a sample from the esophagus of the subject can be processed to isolate and/or purify DNA from the sample. In some embodiments, DNA isolation and/or purification can include cell lysis (e.g., using detergents and/or surfactants). In some embodiments, further processing of DNA (e.g., an amplification reaction) is performed without purifying DNA from the cell lysis. In such cases, additional reagents are added to facilitate further processing including, without limitation, protease inhibitors. In some embodiments, DNA isolation and/or purification can include removing proteins (e.g., using a protease). In some cases, DNA isolation and/or purification can include removing RNA (e.g., using an RNase). In some embodiments, DNA isolation is performed using commercially available kits (for example, without limitation, Qiagen DNAeasy kit) or buffers known in the art (e.g., detergents in Tris-buffer). In some embodiments, the amount DNA inputted (“input DNA”) into the isolation and/or purification reaction may vary depending on a variety of factors including, without limitation, average length of DNA fragments, overall DNA quality, and/or type of DNA (e.g., gDNA, mitochondrial DNA, cfDNA). In some embodiments, any suitable amount of input DNA can be used in the methods described herein.

In some embodiments, the amount of input DNA can be any amount from 1 picogram (pg) to 500 pg. In some embodiments, the amount of input DNA can be at least 0.01 pg, at least 0.01 pg, at least 0.1 pg or at least 1 pg. In some embodiments, the amount of input DNA can be at least 1 picogram (pg), at least 2 pg, at least 3 pg, at least 4 pg, at least 5 pg, at least 6 pg, at least 7 pg, at least 8 pg, at least 9 pg at least 10 pg, at least 11 pg, at least 12 pg, at least 13 pg, at least 14 pg, at least 15 pg, at least 16 pg, at least 17 pg, at least 18 pg, at least 19 pg, at least 20 pg, at least 21 pg, at least 22 pg, at least 23 pg, at least 24 pg, at least 25 pg, at least 26 pg, at least 27 pg, at least 28 pg, at least 29 pg, at least 30 pg, at least 31 pg, at least 32 pg, at least 33 pg, at least 34 pg, at least 35 pg, at least 36 pg, at least 37 pg, at least 38 pg, at least 39 pg or at least 40 pg. In some embodiments, the amount of input DNA is 3 pg.

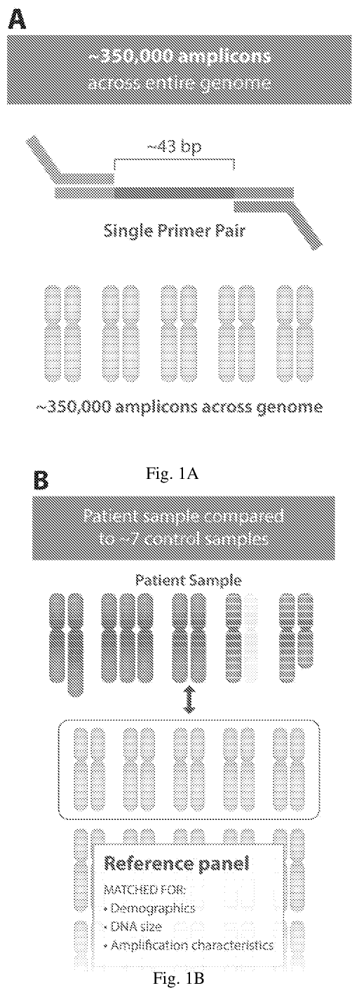

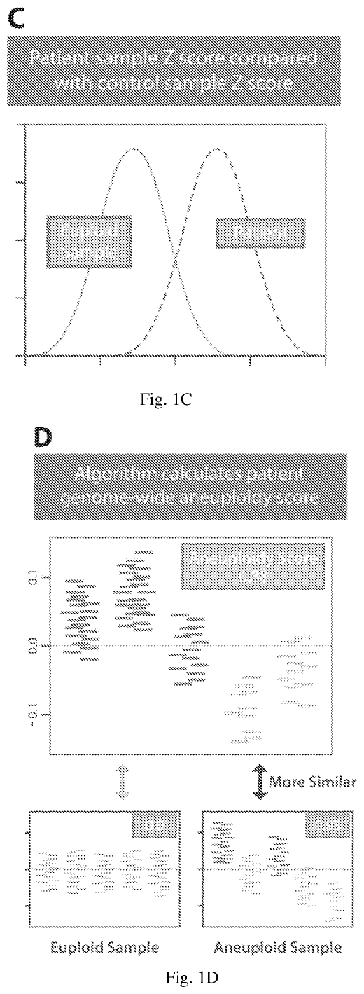

FIG. 1 illustrates a schematic showing Repetitive Element Aneuploidy Sequencing System (RealSeqS) methodology for identifying one or more chromosomal anomalies (e.g., aneuploidies) in the biological sample. The RealSeqS methodology can include amplification of a plurality of amplicons. In some embodiments, the plurality of amplicons are amplified from a plurality of chromosomal sequences in a DNA sample from the biological sample of the esophagus. In some embodiments, the plurality of amplicons can be amplified from any variety of repetitive elements. In some embodiments, the plurality of amplicons is amplified from a plurality of short, interspersed nucleotide elements (SINEs). In some embodiments, the plurality of amplicons can be amplified from a plurality of long interspersed nucleotide elements (LINEs). Methods of amplifying a plurality of amplicons include, without limitation, the polymerase chain reaction (PCR) and isothermal amplification methods (e.g., rolling circle amplification or bridge amplification). In some embodiments, a second amplification step is performed.

In some embodiments, the amplified DNA from a first amplification reaction is used as a template in a second amplification reaction. In some embodiments, the amplified DNA is purified before the second amplification reaction (e.g., PCR purification using methods known in the art).

In some embodiments, an amplification reaction includes using a single pair of primers comprising a first primer having or including SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9. In some embodiments, an amplification reaction includes using a single pair of primers comprising a first primer having at least 80% (e.g., at least 85%, at least 90%, at least 95%, at least 96%, at least 97%, at least 98%, or at least 99%) sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9. In some embodiments, an amplification reaction includes using a single pair of primers comprising a second primer having or including SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18 or SEQ ID NO: 19. In some embodiments, an amplification reaction includes using a single pair of primers comprising a second primer having at least 80% (e.g., at least 85%, at least 90%, at least 95%, at least 96%, at least 97%, at least 98%, or at least 99%) sequence identity to SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18 or SEQ ID NO: 19. In some embodiments, the first primer has a sequence that is at least 80% identical (e.g., at least 85%, at least 90%, at least 95% at least 99%, or 100% identical) to CGACGTAAAACGACGGCCAGTNNNNNNNNNNNNNNNNGGTGAAACCCCGTCTC TACA (SEQ ID NO: 1). In some embodiments, the second primer has a sequence that is at least 80% identical (e.g., at least 85%, at least 90%, at least 95% at least 99%, or 100% identical) to CACACAGGAAACAGCTATGACCATGCCTCCTAAGTAGCTGGGACTACAG (SEQ ID NO: 10). In some embodiments, an amplification reaction includes using a single pair of primers comprising a first primer having SEQ ID NO. 1 and a second primer having SEQ ID NO. 10. In some embodiments, an amplification reaction includes using a single pair of primers comprising a first primer having at least 80% (e.g., at least 85%, at least 90%, at least 95%, at least 96%, at least 97%, at least 98%, or at least 99%) sequence identity to SEQ ID NO. 1 and a second primer having at least 80% (e.g., at least 85%, at least 90%, at least 95%, at least 96%, at least 97%, at least 98%, or at least 99%) sequence identity to SEQ ID NO. 10.

In some embodiments, the first primer comprises from the 5′ to 3′ end: a universal primer sequence (UPS), a unique identifier DNA sequence (UID), and an amplification sequence. In some embodiments, the first primer comprises from the 5′ to 3′ end: a UPS sequence and an amplification sequence. In some embodiments, the first primer comprises from the 5′ to 3′ end: an amplification sequence. In such cases in which the first primer comprises at least an amplification sequence, any variety of library generation techniques known in the art can be used to generate a next generation sequencing library from the amplified amplicons. In some embodiments, the universal primer sequence (UPS) facilitates the generation of a library of amplicons ready for next generation sequencing. For example, an amplicon generated during the amplification reaction using a first primer (SEQ ID NO. 1) and a second primer (SEQ ID NO. 10) is used as a template for a second amplification reaction. In such cases, a second set of primers designed to bind to the UPS includes the 5′ grafting sequences necessary for hybridization to an Illumina flow cell.

In some embodiments, the UID comprises a sequence of 16-20 degenerate bases. In some embodiments, a degenerate sequence is a sequence in which some positions of a nucleotide sequence contain a number of possible bases. In some embodiments of any of the methods described herein, a degenerate sequence can be a degenerate nucleotide sequence comprising about or at least 5, 6, 7, 8, 9, 10, 11, 12, 13, 14, 15, 16, 17, 18, 19, 20, 25, 30, 35, 40, 45, or 50 nucleotides. In some embodiments, a nucleotide sequence contains 1, 2, 3, 4, 5, 6, 7, 8, 9, 0, 10, 15, 20, 25, or more degenerate positions within the nucleotide sequence. In some embodiments, the degenerate sequence is used as a unique identifier DNA sequence (UID). In some embodiments, the degenerate sequence is used to improve the amplification of an amplicon. For example, a degenerate sequence may contain bases complementary to a chromosomal sequence being amplified. In such cases, the increased complementarity may increase a primers affinity for the chromosomal sequence. In some embodiments, the UID (e.g., degenerate bases) is designed to increase a primers affinity to a plurality of chromosomal sequences.

In some embodiments, an amplification reaction includes one or more pairs of primers. In some embodiments, an amplification reaction includes at least 1, at least 2, at least 3, at least 4, at least 5, at least 6, at least 7, at least 8, or at least 9 pairs of primers. In some embodiments, when an amplification reaction includes more than one pair or primers, at least one pair of primers includes a primer having SEQ ID NO: 1 as a first primer and a primer having SEQ ID NO: 10 as a second primer.

In some embodiments, when an amplification reaction includes more than one pair of primers, at least one pair of primers includes a first primer with a sequence having at least 80% (e.g., at least 85%, at least 90%, at least 95%, at least 96%, at least 97%, at least 98%, or at least 99%) sequence identity to SEQ ID NO: 1 and a second primer with a sequence having at least 80% (e.g., at least 85%, at least 90%, at least 95%, at least 96%, at least 97%, at least 98%, or at least 99%) sequence identity to SEQ ID NO: 10.

In some embodiments when an amplification reaction includes one or more pairs of primers, any variety of combinations of primers or pairs of primers can be selected from Table 1. For example, an amplification reaction containing 2 pairs of primers can include a first pair of primers that includes a first primer (e.g., a first primer having SEQ ID NO: 1) and a second primer (e.g., a second primer having SEQ ID NO: 10) and a second pair of primers that includes a third primer (e.g., a third primer having SEQ ID NO: 2) and a fourth primer (e.g., a fourth primer having SEQ ID NO: 11). Combining any of the forward primers (e.g., a “FP” having SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9) with any of the reverse primers (e.g., a “RP” having SEQ ID NO: 10, SEQ ID NO: 11, SEQ ID NO: 12, SEQ ID NO: 14, SEQ ID NO: 15, SEQ ID NO: 16, SEQ ID NO: 17, SEQ ID NO: 18 or SEQ ID NO: 19) will generate amplicons from the repetitive elements as described herein. For example, an amplification reaction containing 2 pairs of primers can include a first pair of primers that includes a first primer (e.g., a first primer having SEQ ID NO: 1) and a second primer (e.g., a second primer having SEQ ID NO: 10) and a second pair of primers that includes a third primer (e.g., a third primer having SEQ ID NO: 2) and a fourth primer (e.g., a fourth primer having SEQ ID NO: 12). In some embodiments, an amplification reaction includes one or more pairs of primers where a first primer is included in both pairs of primers. For example, an amplification reaction can include a first pair of primers that includes a first primer (e.g., a first primer having SEQ ID NO: 1) and a second primer (e.g., a second primer having SEQ ID NO: 10) and a second pair of primers that includes a third primer (e.g., a third primer having SEQ ID NO: 1) and a fourth primer (e.g., a fourth primer having SEQ ID NO: 11).

In some embodiments, a pair of primers are complementary to a plurality of chromosomal sequences. As used herein, the term “complementary” or “complementarity” refers to nucleic acid residues that are capable or participating in Watson-Crick type or analogous base pair interactions that is enough to support amplification. In some embodiments, an amplification sequence of a first primer is designed to amplify one or more chromosomal sequences. In some embodiments, the one or more chromosomal sequence can include any of a variety of repetitive elements as described herein. In some embodiments, the chromosomal sequences are SINEs. In some embodiments, the chromosomal sequences are LINEs. In some embodiments, the chromosomal sequences are a mixture of different types of repetitive elements. In some embodiments when an amplification reaction includes two or more pairs of primers, each pair of primers amplifies a different type of repetitive element. For example, a first pair of primers can amplify SINEs, and a second pair of primers can amplify LINEs. Optionally, a third, fourth, fifth, 5 etc. pair of primers can amplify a third, fourth, fifth, etc. type of repetitive element. In some embodiments when an amplification reaction includes two or more pairs of primers, each pair of primers generates amplicons from the same type of repetitive element. For example, a first pair of primers can amplify SINEs, and 10 a second pair of primers amplify SINEs. Optionally, a third, fourth, fifth, etc. pair of primers can amplify SINEs. In some embodiments when an amplification reaction includes two or more primer pairs, each pair of primers generates amplicons from a mixture of different types of repetitive elements.

In some embodiments, one or both primers of a primer pair described herein include primer modifications. Examples of primer modifications include, without limitation, a spacer (e.g., C3 spacer, PC spacer, hexanediol, spacer 9, spacer 18, l′,2′-dideoxyribose (dspacer)), phosphorylation, phosphorothioate bond modifications, modified nucleic acids, attachment chemistry and/or linker modifications. Examples of modified nucleic acids include, without limitation, 2-Aminopurine, 2,6-Diaminopurine (2-Amino-dA), 5-Bromo dU, deoxyUridine, Inverted dT, Inverted Dideoxy-T, Dideoxy-C, 5-Methyl dC, deoxyinosine, Super T®, Super G®, Locked Nucleic Acids (LNA's), 5-Nitroindole, 2′-O-Methyl RNA Bases, Hydroxymethyl dC, Iso-dQ Iso-dC, Fluoro C, Fluoro U, Fluoro A, Fluoro G, 2-MethoxyEthoxy A, 2-MethoxyEthoxy MeC, 2-MethoxyEthoxy G, and/or 2-MethoxyEthoxy T. Examples of attachment chemistries and linker modifications include, without limitation, Acrydite™, Adenylation, Azide (NHS Ester), Digoxigenin (NHS Ester), Cholesterol-TEG I-Linker, Amino Modifiers (e g, amino modifier C6, amino nodifier C12, amino modifier C6 dT, amino modifier, and/or Uni-Link™ amino modifier), Alkynes (e.g., 5′ Hexynyl and/or 5-Octadiynyl dU), Biotinylation (e.g., biotin, biotin (Azide), biotin dT, biotin-TEQ dual biotin, pC biotin, and/or desthiobiotin-TEG), and/or Thiol Modifications (e.g., thiol modifier C3 S—S, dithiol, and/or thiol modifier C6 S—S).

In some embodiments, any primer as described herein includes synthetic nucleic acids. In some embodiments, one or both primers of a primer pair described herein include primer modifications that enhance processing of amplified DNA. In some embodiments, any primer as described herein includes primer modifications that facilitate elimination of primers (e.g., elimination of primers following an amplification reaction). In some embodiments, primer modifications are conveyed to a product of an amplification reaction (e.g., an amplification product contains modified bases). In such cases, the amplification product includes the modification and the inherent properties of the modification (e.g., the ability to select the amplification product containing the modification).

In some embodiments, methods for identifying one or more chromosomal anomalies as described herein include using amplicon-based sequencing reads. In some embodiments, a plurality of amplicons (e.g., amplicons obtained from a DNA sample) are sequenced. In some embodiments, each amplicon is sequenced at least 1, 2, 3, 4, 5, 6, 7, 8, 9, 10, 11, 12, 13, 14, 15, 16, 17, 18, 19, 20, or more times. In some embodiments, each amplicon can be sequenced between about 1 and about 20 (e.g., between about 1 and about 15, between about 1 and about 12, between about 1 and about 10, between about 1 and about 8, between about 1 and about 5, between about 5 and about 20, between about 7 and about 20, between about 10 and about 20, between about 13 and about 20, between about 3 and about 18, between about 5 and about 16, or between about 8 and about 12) times. In some cases, amplicon-based sequencing reads can include continuous sequencing reads. In some cases, amplicons include short, interspersed nucleotide elements (SINEs).

In some cases, amplicon-based sequencing reads can include from about 100,000 to about 25 million (e.g., from about 100,000 to about 20 million, from about 100,000 to about 15 million, from about 100,000 to about 12 million, from about 100,000 to about 10 million, from about 100,000 to about 5 million, from about 100,000 to about 1 million, from about 100,000 to about 750,000, from about 100,000 to about 500,000, from about 100,000 to about 250,000, from about 250,000 to about 25 million, from about 500,000 to about 25 million, from about 750,000 to about 25 million, from about 1 million to about 25 million, from about 5 million to about 25 million, from about 10 million to about 25 million, from about 15 million to about 25 million, from about 200,000 to about 20 million, from about 250,000 to about 15 million, from about 500,000 to about 10 million, from about 750,000 to about 5 million, or from about 1 million to about 2 million) sequencing reads. For example, sequencing a plurality of amplicons can include assigning a unique identifier (UID) to each template molecule (e.g., to each amplicon), amplifying each uniquely tagged template molecule to create UID-families, and redundantly sequencing the amplification products.

In some embodiments, sequencing a plurality of amplicons can include calculating a Z-score of a variant on said selected chromosome arm using the equation

$$Z ∼ \frac{{\sum }_{i}^{k} = {w}_{i} {Z}_{i}}{\sqrt{{\sum }_{i}^{k} {w}_{i}^{σ}}}$$

where wᵢ, is UID depth at a variant i, Zi is the Z-score of variant i, and k is the number of variants observed on the chromosome arm, which is described, for example, WO 2020/236625 A2, which is herein incorporated by reference in its entirety. In some embodiments, methods of sequencing amplicons can include methods known in the art (see, e.g., US Pat. No. 2015/0051085; and Kinde et al. 2012 PloS ONE 7: e41162, which are herein incorporated by reference in their entireties).

In some embodiments, amplicons are aligned to a reference genome. In some embodiments, a plurality of amplicons generated by methods described herein includes from about 10,000 to about 1,000,000 (e.g., from about 15,000 to about 1,000,000, from about 25,000 to about 1,000,000, from about 35,000 to about 1,000,000, from about 50,000 to about 1,000,000, from about 75,000 to about 1,000,000, from about 100,000 to about 1,000,000, from about 125,000 to about 1,000,000, from about 160,000 to about 1,000,000, from about 180,000 to about 1,000,000, from about 200,000 to about 1,000,000, from about 300,000 to about 1,000,000, from about 500,000 to about 1,000,000, from about 750,000 to about 1,000,000, from about 10,000 to about 800,000, from about 10,000 to about 500,000, from about 10,000 to about 250,000, from about 10,000 to about 150,000, from about 10,000 to about 100,000, from about 10,000 to about 75,000, from about 10,000 to about 50,000, from about 10,000 to about 40,000, from about 10,000 to about 30,000, or from about 10,000 to about 20,000) amplicons (e.g., unique amplicons).

As one non-limiting example, a plurality of amplicons can include about 745,000 amplicons (e.g., 745,000 unique amplicons) Amplicons in a plurality of amplicons can include from about 30 to about 140 (e.g., from about 30 to about 140, from about 40 to about 140, from about 90 to about 140, from about 100 to about 140, from about 130 to about 140, from about 30 to about 130, from about 30 to about 120, from about 30 to about 110, from about 30 to about 100, from about 30 to about 90, from about 30 to about 80, from about 60 to about 130, from about 70 to about 125, from about 80 to about 120, or from about 90 to about 100) nucleotides. As one non-limiting example, an amplicon can include about 100 nucleotides.

In some embodiments, methods and materials for identifying one or more chromosomal anomalies as described herein include grouping sequencing reads (e.g., from a plurality of amplicons) into clusters (e.g., unique clusters) of genomic intervals. In some embodiments, a genomic interval is included in one or more clusters. In some embodiments, a genomic interval can belong to from about 100 to about 252 (e.g., from about 125 to about 252, from about 150 to about 252, from about 175 to about 252, from about 200 to about 252, from about 225 to about 252, from about 100 to about 250, from about 100 to about 225, from about 100 to about 200, from about 100 to about 175, from about 100 to about 150, from about 125 to about 225, from about 150 to about 200, or from about 160 to about 180) clusters. As one non-limiting example, a genomic interval can belong to about 176 clusters.

In some embodiments, each cluster includes any appropriate number of genomic intervals. In some embodiments, each cluster includes the same number of genomic intervals. In some embodiments, different clusters include varying numbers of genomic clusters. As one nonlimiting example, each cluster can include about 200 genomic intervals. In some embodiments, genomic intervals are identified as having shared amplicon features. As used herein, the term “shared amplicon feature” refers to amplicons with one or more features that are similar. In some embodiments, a plurality of genomic intervals are grouped into a cluster based on one or more shared amplicon features of the sequencing reads mapped to a genomic interval. In some embodiments, the shared amplicon feature is the number amplicons mapped to a genomic interval (e.g., sums of the distributions of the sequencing reads in each genomic interval). In some embodiments, the shared amplicon feature is the average length of the mapped amplicons.

In some embodiments, a cluster of genomic intervals includes from about 5000 to about 6000 (e.g., from about 5100 to about 6000, from about 5200 to about 6000, from about 5300 to about 6000, from about 5400 to about 6000, from about 5500 to about 6000, from about 5600 to about 6000, from about 5700 to about 6000, from about 5800 to about 6000, from about 5900 to about 6000, from about 5000 to about 5900, from about 5000 to about 5800, from about 5000 to about 5700, from about 5000 to about 5600, from about 5000 to about 5500, from about 5000 to about 5400, from about 5000 to about 5300, from about 5000 to about 5200, from about 5000 to about 5100, from about 5100 to about 5800, from about 5100 to about 5700, from about 5100 to about 5600, from about 5100 to about 5500, from about 5100 to about 5400, from about 5100 to about 5300, from about 5100 to about 5200, from about 5200 to about 5600, from about 5200 to about 5500, from about 5200 to about 5400, from about 5200 to about 5300, from about 5300 to about 5500, from about 5300 to about 5400, or from about 5400 to 5500 from about 5200 to about 5700, or from about 5300 to about 5500) genomic intervals.

As one non-limiting example, a cluster of genomic intervals can include about 5344 genomic intervals. A genomic interval can be any appropriate length. For example, a genomic interval can be the length of an amplicon sequenced as described herein. For example, a genomic interval can be the length of a chromosome arm. In some cases, a genomic interval can include from about 100 to about 125,000,000 (e.g., from about 250 to about 125,000,000, from about 500 to about 125,000,000, from about 750 to about 125,000,000, from about 1,000 to about 125,000,000, from about 1,500 to about 125,000,000, from about 2,000 to about 125,000,000, from about 5,000 to about 125,000,000, from about 7,500 to about 125,000,000, from about 10,000 to about 125,000,000, from about 25,000 to about 125,000,000, from about 50,000 to about 125,000,000, from about 100,000 to about 125,000,000, from about 250,000 to about 125,000,000, from about 500,000 to about 125,000,000, from about 100 to about 1,000,000, from about 100 to about 750,000, from about 100 to about 500,000, from about 100 to about 250,000, from about 100 to about 100,000, from about 100 to about 50,000, from about 100 to about 25,000, from about 100 to about 10,000, from about 100 to about 5,000, from about 100 to about 2,500, from about 100 to about 1,000, from about 100 to about 750, from about 100 to about 500, from about 100 to about 250, from about 500 to about 1,000,000, from about 5000 to about 900,000, from about 50,000 to about 800,000, or from about 100,000 to about 750,000) nucleotides. As one non-limiting example, a genomic interval can include about 500,000 nucleotides.

In some embodiments, clusters of genomic intervals are formed using any appropriate method known in the art. In some embodiments, clusters of genomic intervals are formed based on shared amplicon features of the genomic intervals (see, e.g., Douville et al. PNAS 201 115(8):1871-1876, which is herein incorporated by reference in its entirety). In some embodiments, methods and materials described herein for identifying one or more chromosomal anomalies include assessing a genome (e.g., a genome of a mammal) for the presence or absence of one or more chromosomal anomalies (e.g., aneuploidies). The presence or absence of one or more chromosomal anomalies in the genome of a mammal can, for example, be determined by sequencing a plurality of amplicons obtained from a sample (e.g., a test sample) obtained from the mammal to obtain sequencing reads, and grouping the sequencing reads into clusters of genomic intervals. In some cases, read counts of genomic intervals can be compared to read counts of other genomic intervals within the same sample. In some cases where read counts of genomic intervals are compared to read counts of other genomic intervals within the same sample, a second (e.g., control or reference) sample is not assayed. In some cases, read counts of genomic intervals can be compared to read counts of genomic intervals in another sample. For example, when using methods and materials described herein to identify genetic relatedness, polymorphisms (e.g., somatic mutations), and/or microsatellite instability, genomic intervals can be compared to read counts of genomic intervals in a reference sample. A reference sample can be a synthetic sample. A reference sample can be from a database. In some cases where methods and materials described herein are used to identify anomalies (e.g., aneuploidies), a reference sample can be a normal sample obtained from the same cancer patient (e.g., a sample from the cancer patient that does not harbor cancer cells) or a normal sample from another source (e.g., a patient that does not have cancer). In some cases where method and materials described herein are used to identify anomalies (e.g., aneuploidies), a reference sample can be a normal sample obtained from the same patient (e.g., a sample from pre-natal human that contains only maternal cells).

In some embodiments, methods and materials described herein are used for detecting aneuploidy in a genome of a mammal having or suspected of having BE. For example, a plurality of amplicons obtained from a sample obtained from a mammal can be sequenced, the sequencing reads can be grouped into clusters of genomic intervals, the sums of the distributions of the sequencing reads in each genomic interval can be calculated, a Z-score of a chromosome arm can be calculated, and the presence or absence of an aneuploidy in the genome of the mammal can be identified.

The distributions of the sequencing reads in each genomic interval can be summed. For example, sums of distributions of the sequencing reads in each genomic interval can be calculated using the equation,

Σ₁^{I}Rᵢ˜N(Σ₁^{I}μᵢΣ₁^{I}σᵢ²)
where Ri is the number of sequencing reads, I is the number of clusters on a chromosome arm, N is a Gaussian distribution with parameters μᵢ and σᵢ², μᵢ is the mean number of sequencing reads in each genomic interval, and σᵢ² is the variance of sequencing reads in each genomic interval. A Z-score of a chromosome arm can be calculated using any appropriate technique. For example, a Z-score of a chromosome arm can be calculated using the quantile function 1-CDF(Σ₁^{I}μᵢΣ₁^{I}σᵢ²).

The presence of an aneuploidy in the genome of the mammal can be identified in the genome of the mammal when the Z-score is outside a predetermined significance threshold, and the absence of an aneuploidy in the genome of the mammal can be identified in the genome of the mammal when the Z-score is within a predetermined significance threshold. The predetermined threshold can correspond to the confidence in the test and the acceptable number of false positives. For example, a significance threshold can be ±2 or ±3.

In some embodiments, chromosomal gains are identified by any of values of Z_{w} (also denoted as Z) of >2.0, >2.1>, >2.2, >2.3, >2.4>, >2.5, >2.6, >2.7, >2.8, >2.9, >3.0, or by a value exceeding a cutoff between 2.0 to 3.0, and chromosomal losses are identified by any of values of Z_{w} (also denoted as Z) of <−2.0, <−2.1, <−2.2, <−2.3, <−2.4, <−2.5, <−2.6, <−2.7, <−2.8, <−2.9, <−3.0, or by a value lower than a cutoff between −2.0 to −3.0.

In some embodiments, methods and materials described herein employ supervised machine learning. In some embodiments, supervised machine learning can detect small changes in one or more non-acrocentric chromosome arms. For example, supervised machine learning can detect changes such as chromosome arm gains or losses that are often present in cancer associated with chromosomal anomalies, such as esophageal cancer. In some embodiments, supervised machine learning can detect changes such as chromosome arm gains or losses that are present in a biological sample form the esophagus of a subject. In some cases, supervised machine learning can be used to classify samples according to aneuploidy status. For example, supervised machine learning can be employed to make genome-wide aneuploidy calls. In some cases, a support vector machine model can include obtaining an SVM score. An SVM score can be obtained using any appropriate technique. In some cases, an SVM score can be obtained as described elsewhere (see, e.g., Cortes 1995 Machine learning 20:273-297; and Meyer et al. 2015 R package version: 1.6-3). At lower read depths, a sample will typically have a higher raw SVM score. Thus, in some cases, raw SVM probabilities can be corrected based on the read depth of a sample using the equation

$$log ( 1 - \frac{1}{r} ) = A x + B$$

where r is the ratio of the SVM score at a particular read depth/minimum SVM score of a particular sample given sufficient read depth. A and B can be determined as described in WO 2020/236625 A2, which is herein incorporated by reference in its entirety.

Also provided herein are new methods of normalization that reduce the amount of variability between samples. In some embodiments, a principal component analysis (PCA) can be used for normalization. In some embodiments, a PCA is performed on sequencing data from the controls. For example, a PCA may reduce the number of 500 kb genomic intervals to a more manageable number of dimensions. Using the PCA coordinates of the controls, a model can be generated that predicts whether a particular 500 kb interval will be amplified more or less efficiently in future samples based on their PCA coordinates.

Correction Factor for 500 kb Interval,=β₀ᵢ+β₁ᵢ*PCA₂+β₃ᵢ*PCA₃+β₄ᵢ*PCA₄+β₅ᵢ*PCA₅

For example, for each test sample, a sample can be projected into PCA space and the correction factor can be calculated for each 500 kb interval as function of its PCA coordinates. After applying the correction factor to each 500 kb genomic interval, the test sample may be matched to one or more control samples based on the closest Euclidean distance of the 500 kb intervals. In some embodiments, samples are excluded in order to ensure the quality of the data. In some embodiments, samples are excluded before, contemporaneously with, and/or after data analysis. In some embodiments, a list of factors can be applied to the data in order to exclude data that does not meet the criteria set forth in the list of factors. In some embodiments, the list of factors may be any reasonable number of factors. For example, a list of five factors can be used to exclude samples. Any combination of factors can be used to determine that a sample should be excluded. In some embodiments, samples with fewer than 2.5M reads may be excluded. In some embodiments, samples with sufficient evidence of contamination may be excluded. For example, a sample may be considered contaminated if the sample has at least 10 significant allelic imbalanced chromosome arms (z score≥2.0, ≥2.5, or ≥3.0) and fewer than ten significant chromosome arms gains or losses (z≥2.0, ≥2.5, or ≥3.0 or z≤−2.0, ≤−2.5, or ≤−3). In some embodiments, allelic imbalance can be determined from SNPs, while gains or losses can be assessed through Within-Sample AneupLoidy Detection (WALDO) algorithm. In some embodiments, when examining the quality of the samples obtained from the esophagus, samples may be excluded in which a certain percentage of the amplicons are larger than a predetermined number or, for example, 50 base pairs between the forward and reverse primers). Without wishing to be bound by theory, such samples may be contaminated with leukocyte DNA. In some embodiments, samples outside the dynamic range of the assay may be excluded.

In one example, the WALDO algorithm can compare the normalized read counts of 500 kb intervals to intervals on other chromosome arms in the same sample. Its normalization is therefore internal, “within-sample.” The intervals are aggregated across the entire length of the chromosome arm to produce an arm level statistical significance score (Zw). The non-acrocentric Zw values serve as features that are integrated and modeled with a support vector machine (SVM) to provide a summary Global Aneuploidy Score (GAS) that discriminates between aneuploid and euploid samples. The SVM classifier can be trained on normal euploid plasma samples and in silico aneuploid samples generated from the normal plasma samples. The in silico samples can be generated to mimic recurrently altered chromosome arms observed in cancers, including esophageal cancers. The 500 kb clusters used to define aneuploidy in the test sample can be generated from matched esophageal samples. RealSeqS, WALDO, and the SVM classifier can all be performed by investigators blinded to the clinical classification of the associated samples. The software used to generate the scores reported in this manuscript are available (https://doi.org/10.5281/zenodo.3656943)

In some embodiments, a GAS generated using the Z_{w} and SWM that is indicative of presence of dysplasia or cancer, or increased risk of progression to low grade dysplasia, or high grade dysplasia, or cancer, is >0.1, or >0.2, or >0.3, or >0.4, or >0.6, or >0.8, or >0.9, or >0.907, preferably, >0.6, or >0.8, or >0.9, or >0.907. In other embodiments, a GAS indicative of presence of dysplasia or cancer, or increased risk of progression to low grade dysplasia, or high grade dysplasia, or cancer is >0.1, or >0.2, or >0.3, or >0.4, or >0.6. In still other embodiment, a GAS indicative of normal esophagus or NDBE is ≤0.1, or ≤0.2, or ≤0.3, or ≤0.4, or ≤0.6.

In some embodiments, the GAS score can be combined with data on specific chromosome alterations in the biological sample generated using a circular binary segmentation algorithm to provide a Barrett's Aneuploidy Decision (BAD) classifier for distinguishing stages of BE progression. The chromosomal alterations generated using a circular binary segmentation algorithm indicative of BE progression can include chromosome gains and/or chromosome losses in non-acrocentric chromosomes of the subject. For example, the chromosomal alterations can include chromosomal gains of any of chromosome regions 8q24, 1q, 7q, 7p, 20q, 2q, 13q, 5p, or 12p and/or losses of any of chromosome regions 5q, 17p, 4p, 4q, 9p, 18q, 16q, 21q, 22q, or 10p. For example, the chromosomal alterations can include gains of any of chromosome regions 1q, 12p, 8q24, or 20q, and/or losses of chromosomes regions 9p or 17p. In other examples, the chromosomal alterations can include gains or losses in any one of 1q, 2q, 4q, 5q, 7p, 7q, 9p, 12p, 17p, or 20q.

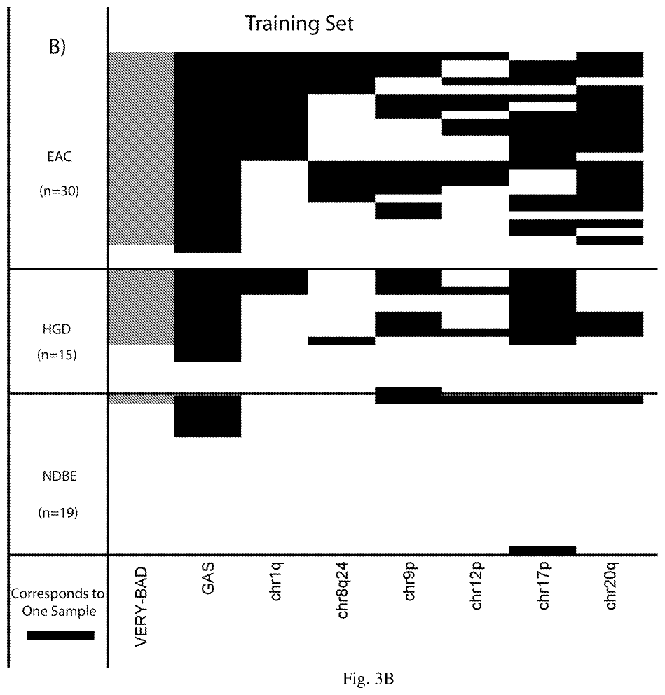

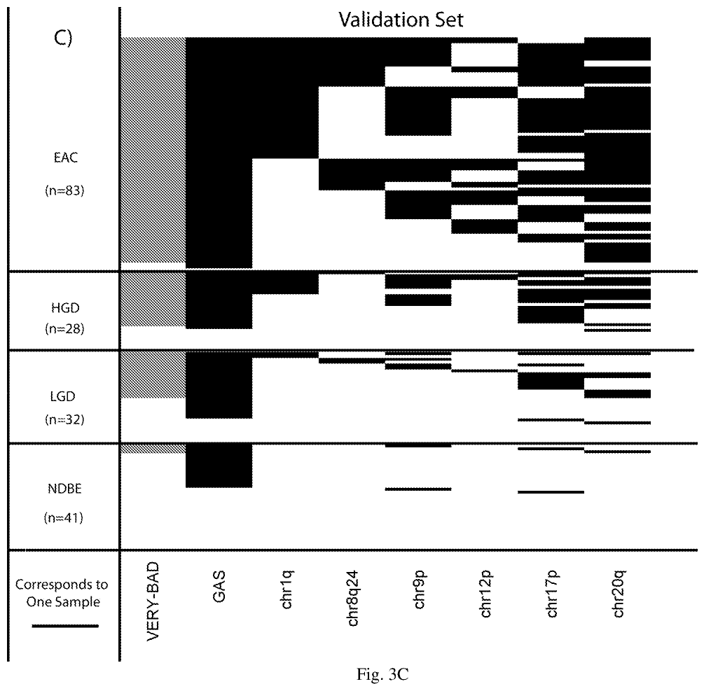

In some embodiments, BAD can be used to sort samples into three categories based on the measured GAS and specific chromosome alterations. Not-BAD cases can have a GAS≤0.1, or ≤0.2, or ≤0.3, or ≤0.4, or ≤0.6, indicating relative non-aneuploidy. Maybe-BAD cases can have a GAS>0.1, or >0.2, or >0.3, or >0.4, or >0.6 but none of the specific chromosome alterations, indicating a greater potential risk of progression. Very-BAD cases can have GAS>0.1, or >0.2, or >0.3, or >0.4, or >0.6 and losses of 9p or 17p, gains of 1q, 12p, or 20q, or a focal gain of 8q24 (FIG. 3A, 3B, Table 8).

The BAD classification system, which used both specific chromosome changes plus GAS scores, can outperform GAS scores alone. In particular, compared to the aneuploidy classification of GAS, the Very-BAD classification markedly improved both the specificity for rejecting NDBE and the positive predictive value (PPV) for identifying HGD plus EAC cases. Moreover, compared to aneuploidy alone, the Very-BAD classification produced only minimal decreases in the sensitivity for detecting HGD or EAC or in the negative predictive value (FIG. 3B, Table 7). NDBE cases classified as Very-BAD may benefit from intensified surveillance, while the Not-BAD cases may require less surveillance. Similarly, the LGD cases classified as Very-BAD may benefit from ablation therapies, whereas Not-BAD cases could potentially be followed with continued endoscopic surveillance.

In some embodiments, the methods described herein can be used to detect Barrett's esophagus (BE) with low grade dysplasia (LGD), or Barrett's esophagus (BE) with high grade dysplasia (HGD), or adenocarcinoma of the esophagus (EAC) in subject having or suspected of having BE or increased progression to LGD, HGD, or EAC in a subject with BE. In some embodiments, the subject may be undergoing routine screening and may not necessarily be suspected of having such metaplasia or neoplasia. In some embodiments, a subject is determined to be prone to developing and/or has developed a BE with LGD, BE with HGD, or AEC or has an increased progression to LGD, HGD, or EAC if a biological sample obtained from the subject has a GAS of >0.1, or >0.2, or >0.3, or >0.4, or >0.6, or >0.8, or >0.9, or >0.907 and a chromosomal alteration in at least one of 8q24, 1q, 7q, 7p, 20q, 2q, 13q, 5p, 12p, 5q, 17p, 4p, 4q, 9p, 18q, 16q, 21q, 22q, or 10p. In some embodiments, the chromosomal alterations can include gains of any of chromosome regions 8q24, 1q, 7q, 7p, 20q, 2q, 13q, 5p, or 12p and/or losses of any of chromosome regions 5q, 17p, 4p, 4q, 9p, 18q, 16q, 21q, 22q, or 10p. For example, the chromosomal alterations can include gains of any of chromosome regions 1q, 12p, 8q24, or 20q, and/or losses of chromosomes regions 9p or 17p. In other examples, the chromosomal alterations can include gains or losses in any one of 1q, 2q, 4q, 5q, 7p, 7q, 9p, 12p, 17p, or 20q.

In some embodiments, chromosomal gains are identified by any of values of Z_{w} (also denoted as Z) of >2.0, >2.1>, >2.2, >2.3, >2.4>, >2.5, >2.6, >2.7, >2.8, >2.9, >3.0, or by a value exceeding a cutoff between 2.0 to 3.0, and chromosomal losses are identified by any of values of Z_{w} (also denoted as Z) of <−2.0, <−2.1, <−2.2, <−2.3, <−2.4, <−2.5, <−2.6, <−2.7, <−2.8, <−2.9, <−3.0, or by a value lower than a cutoff between −2.0 to −3.0.

In other embodiments, the subject is prone to developing and/or has developed a BE with LGD, BE with HGD, or AEC or has an increased progression to LGD, HGD, or EAC if the determined GAS of the biological sample obtained from the subject is >0.6, and a chromosomal alteration in at least one of 8q24, 1q, 7q, 7p, 20q, 2q, 13q, 5p, 12p, 5q, 17p, 4p, 4q, 9p, 18q, 16q, 21q, 22q, or 10p. In some embodiments, the chromosomal alterations can include gains of any of chromosome regions 8q24, 1q, 7q, 7p, 20q, 2q, 13q, 5p, or 12p and/or losses of any of chromosome regions 5q, 17p, 4p, 4q, 9p, 18q, 16q, 21q, 22q, or 10p. For example, the chromosomal alterations can include gains of any of chromosome regions 1q, 12p, 8q24, or 20q, and/or losses of chromosomes regions 9p or 17p. In other examples, the chromosomal alterations can include gains or losses in any one of 1q, 2q, 4q, 5q, 7p, 7q, 9p, 12p, 17p, or 20q.

In some embodiments, chromosomal gains are identified by any of values of Z_{w} (also denoted as Z) of >2.0, >2.1>, >2.2, >2.3, >2.4>, >2.5, >2.6, >2.7, >2.8, >2.9, >3.0, or by a value exceeding a cutoff between 2.0 to 3.0, and chromosomal losses are identified by any of values of Z_{w} (also denoted as Z) of <−2.0, <−2.1, <−2.2, <−2.3, <−2.4, <−2.5, <−2.6, <−2.7, <−2.8, <−2.9, <−3.0, or by a value lower than a cutoff between −2.0 to −3.0.

In some embodiments, if the subject is determined to have an BE with LGD, BE with HGD, or EAC, the subject may be administered any of cryotherapy, photodynamic therapy (PDT); radiofrequency ablation (RFA); laser ablation; argon plasma coagulation (APC); electrocoagulation (electrofulguration); esophageal stent, surgery, and/or a therapeutic agent.

In other embodiments, the subject has BE with LGD and an increased progression to HGD or EAC if the determined GAS of the biological sample obtained from the subject is >0.6, and a chromosomal alteration in at least one of 8q24, 1q, 7q, 7p, 20q, 2q, 13q, 5p, 12p, 5q, 17p, 4p, 4q, 9p, 18q, 16q, 21q, 22q, or 10p. In some embodiments, the chromosomal alterations can include gains of any of chromosome regions 8q24, 1q, 7q, 7p, 20q, 2q, 13q, 5p, or 12p and/or losses of any of chromosome regions 5q, 17p, 4p, 4q, 9p, 18q, 16q, 21q, 22q, or 10p. For example, the chromosomal alterations can include gains of any of chromosome regions 1q, 12p, 8q24, or 20q, and/or losses of chromosomes regions 9p or 17p. In other examples, the chromosomal alterations can include gains or losses in any one of 1q, 2q, 4q, 5q, 7p, 7q, 9p, 12p, 17p, or 20q.

In some embodiments, chromosomal gains are identified by any of values of Z_{w} (also denoted as Z) of >2.0, >2.1>, >2.2, >2.3, >2.4>, >2.5, >2.6, >2.7, >2.8, >2.9, >3.0, or by a value exceeding a cutoff between 2.0 to 3.0, and chromosomal losses are identified by any of values of Z_{w} (also denoted as Z) of <−2.0, <−2.1, <−2.2, <−2.3, <−2.4, <−2.5, <−2.6, <−2.7, <−2.8, <−2.9, <−3.0, or by a value lower than a cutoff between −2.0 to −3.0.

In some embodiments, the RealSeqS methodology described herein can employ a computer readable storage medium and a processor (not shown) configured to calculate, compare and/or determine the Z_{w}, GAS, specific chromosomal alterations, and/or BAD and provide real-time feedback to a subject of the results. These results, in turn, can be readily transmitted to a primary care provider and/or stored in a medical record database.

The RealSeqS methodology may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, a computer readable medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) can be encoded with one or more programs that, when executed on one or more computers or other processors, perform methods that implement the various embodiments described herein. The computer readable medium or media can be transportable, such that the program or programs stored thereon can be loaded onto one or more different computers or other processors to implement various aspects described herein. As used herein, the term “non-transitory computer-readable storage medium” encompasses only a computer-readable medium that can be considered to be a manufacture (i.e., article of manufacture) or a machine.

The terms “program” or “software” are used herein in a generic sense to refer to any type of computer code or set of computer-executable instructions that can be employed to program a computer or other processor to implement various aspects as discussed above. Additionally, it should be appreciated that according to one aspect of this embodiment, one or more computer programs that when executed perform methods of described herein need not reside on a single computer or processor, but may be distributed in a modular fashion amongst a number of different computers or processors to implement various aspects herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

The processor can determine Barrett's esophagus (BE) with low grade dysplasia (LGD), or Barrett's esophagus (BE) with high grade dysplasia (HGD), or adenocarcinoma of the esophagus (EAC) and/or BE at increased risk of progression to LGD, or HGD, or EAC based on the GAS, specific chromosomal alterations, and/or BAD of the biological sample obtained from the esophagus.

In some embodiments, if the subject is determined to have an increased progression to HGD or EAC, the subject may be administered any of cryotherapy, photodynamic therapy (PDT); radiofrequency ablation (RFA); laser ablation; argon plasma coagulation (APC); electrocoagulation (electrofulguration); esophageal stent, surgery, and/or a therapeutic agent.

In some embodiments, the disclosure provides for a method of determining whether a biological sample from the esophagus of the subject has a GAS and/or chromosomal alterations that are indicative of BE with LGD, BE with HGD, or AEC or has an increased progression to LGD, HGD, or EAC, wherein if the subject is determined to have a LGD, HGD, or EAC, the subject is treated with an agent that treats the BE with LGD, BE with HGD, or EAC.

In some embodiments, the treatment of a BE with LGD, BE with HGD, or EAC encompasses administration of any one or more of the following compounds: proton pump inhibitors (PPIs), such as omeprazole (Prilosec, Zegerid), lansoprazole (Prevacid), pantoprazole (Protonix), rabeprazole (AcipHex), esomeprazole (Nexium), dexlansoprazole (Dexilant). Histamine H2 receptor blocking agents such as cimetidine (Tagamet), ranitidine (Zantac), famotidine (Pepcid) and nizatidine (Axid). Tums, Rolaids, or other quick-acting reflux medications. Prokinetic agents, or drugs that help move food through the gastrointestinal tract more quickly, offer an attractive alternative either alone or in combination with acid inhibition. In some embodiments, the treatment of a BE is endoscopic mucosal resection (EMR); photodynamic therapy (PDT), radiofrequency ablation (RFA); argon plasma coagulation (APC); cryotherapy, and/or surgery (e.g. esophagectomy, anti-reflux surgery).

In other embodiments, the treatment of a BE with LGD, BE with HGD, or EAC encompasses surgery (e.g., esophagectomy), radiation therapy, chemoradiation therapy and/or chemotherapy. In some embodiments, the treatment of esophageal neoplasia (e.g., esophageal cancer) encompasses administering one or more chemotherapeutic agent, such as any one or more therapeutic agent selected from the group consisting of: carboplatin and paclitaxel (Taxol) (which may be combined with radiation); cisplatin and 5-fluorouracil (5-FU) (often combined with radiation): ECF: epirubicine (Ellence), cisplatin, and 5-FU (especially for gastroesophageal junction tumors); DCF: docetaxel (Taxoteret), cisplatin, and 5-FU; Cisplatin with capecitabine (Xeloda); oxaliplatin and either 5-FU or capecitabine: doxorubicin (Adriamycin), bleomycin, mitomycin, methotrexate, vinorelbine (Navelbine), topotecan, and irinotecan (Camptosar).

The terms “treatment”, “treating”, “alleviation” and the like are used herein to generally mean obtaining a desired pharmacologic and/or physiologic effect, and may also be used to refer to improving, alleviating, and/or decreasing the severity of one or more symptoms of a condition being treated. The effect may be prophylactic in terms of completely or partially delaying the onset or recurrence of a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

Treating a Barrett's esophagus and/or esophageal cancer in a subject refers to improving (improving the subject's condition), alleviating, delaying or slowing progression or onset, decreasing the severity of one or more symptoms associated with Barrett's esophagus and/or an esophageal cancer. For example, treating a metaplasia or neoplasia includes any one or more of: reducing growth, proliferation and/or survival of metaplastic/neoplastic cells, killing metaplastic/neoplastic cells (e.g., by necrosis, apoptosis or autophagy), decreasing metaplasia/neoplasia size, decreasing rate of metaplasia/neoplasia size increase, halting increase in metaplasia/neoplasia size, improving ability to swallow, decreasing internal bleeding, decreasing incidence of vomiting, reducing fatigue, decreasing the number of metastases, decreasing pain, increasing survival, and increasing progression free survival.

It will be appreciated that a mammal identified as having Barrett's esophagus (BE) with low grade dysplasia (LGD), or Barrett's esophagus (BE) with high grade dysplasia (HGD), or adenocarcinoma of the esophagus (EAC); BE at increased risk of progression to LGD, or HGD, or EAC; and/or BE with LGD at increased risk of progression to HGD, or EAC can have the diagnosis confirmed using any appropriate method. Examples of methods that can be used to confirm the presence of one or more chromosomal anomalies include, without limitation, karyotyping, fluorescence in situ hybridization (FISH), quantitative PCR of short tandem repeats, quantitative fluorescence PCR (QF-PCR), quantitative PCR dosage analysis, quantitative mass spectrometry of SNPs, comparative genomic hybridization (CGH), whole genome sequencing, and exome sequencing.

In some embodiments, the method diagnosis can be confirmed by identifying genomic loci (e.g., vimentin and/or SqBE18) that are differentially methylated in BE and EAC. Identification of methylated genomic loci associated with BE and EAC is described, for example, in U.S. Patent Publication No. 2019/0309372, which is incorporated by reference in its entirety.

### EXAMPLE

In this Example, we assessed whether a single esophageal brushing that widely sampled the esophagus could be combined with massively parallel sequencing to characterize aneuploidy and identify patients with disease progression to dysplasia or cancer. Esophageal brushing is a method to conveniently sample an extensive area of esophageal epithelium. However, the cells collected from brushings represent a mixture of normal epithelium, non-dysplastic Barrett's epithelium, and dysplastic epithelium, thereby substantially diluting the genomic signal originating from the dysplastic cells. To evaluate aneuploidy in such brushings, a technique that can sensitively detect aneuploidy in a mixed cellular population is required. The Repetitive Element Sequencing System (RealSeqS) is a recently described massively parallel sequencing (MPS) approach that was designed for the detection of aneuploidy in plasma samples containing low levels of DNA derived from neoplastic cells (FIG. 1).

The implementation of this concept required evaluation of DNA from a relatively large number of esophageal brushings as well as re-normalization of the basic algorithms used to evaluate the RealSeqS data from previous experiments on plasma. In this Example, we describe this implementation and apply it to the evaluation of samples from patients with BE at various stages of disease. The primary aim of the clinical component of this study was to develop a classifier based on RealSeqS data to distinguish the progressive phases of BE. The secondary aim was to identify specific chromosomal alterations that tracked with disease progression.

### Methods

### Patient Samples

The study was cross sectional in design. Patients were recruited prior to esophagogastroduodenoscopy (EGD) as part of an Institutional Review Board-approved, multi-institutional study to develop biomarkers for Barrett's esophagus (BE) and esophageal adenocarcinoma (EAC). The six participating institutions are tertiary centers that care for patients referred for management of dysplastic BE and EAC. Brushings were obtained before any biopsies were taken, but because the study cohort was in general individuals referred for tertiary care, many patients had already obtained a diagnosis. Patients in general underwent sampling and endoscopy on the day they were enrolled in the study. Brushings of patients with BE or LGD were of the entire BE segment, while brushings of patients with known HGD and EAC sampled a 3 cm patch targeted to include any nodularity, depression, or irregularity, as these areas would most likely contain the highest grade lesion. Ninety-nine esophageal brushings from concurrently enrolled subjects without BE were also performed with an approximately 3-cm long brushing obtained that covered the gastroesophageal junction and distal esophagus.

Following retrieval, brushes were cut with scissors into cryovials, snap frozen at bedside, then stored at −80° C.

Seventy-nine patients were sampled in a Training Set and 258 patients in a Validation Set. Duplicate esophageal brushings were obtained at the same clinical session in 22 participants and were used to assess assay concordance.

### Histopathologic Diagnosis

The diagnosis of NDBE, LGD, or HGD was primarily determined by histopathology of the biopsy obtained after brushing at the time of study entry. Slides were retrievable for central pathology review for 76% of the Validation Set cases. For all other study cases, diagnoses were as determined by expert GI pathologists at the respective enrollment centers. The presence of surface epithelium was confirmed on all reviewed biopsies. When biopsies were not performed on the day of the brush sampling, the results of biopsy from EGDs performed within the previous three months were used. For study purposes, cases with pathology described as focal HGD were classified as HGD. Intramucosal cancer was classified as EAC.

### Detection of Chromosome Arm Alterations

A single primer pair was used to amplify ˜350,000 loci spread throughout the genome (FIG. 1). One of the primers included a unique identifier sequence (UID) as a molecular barcode of 16 degenerate bases to reduce error rates associated with sequencing, performed on an Illumina HiSeq 4000. The average number of uniquely aligned reads was 11.2 Million (M) (interquartile range 9.7 M-12.8 M). Sequencing data were processed to identify single chromosomal arm gains or losses using the Within-Sample AneupLoidy Detection (WALDO) algorithm incorporated into the RealSeqS workflow. Fifteen esophageal brushings from individuals without BE were used as reference samples; these were excluded from all other analyses. Each experimental sample was then matched to the reference samples that were most similar to it with respect to the amplicon distributions generated by RealSeqS. The reference samples included four individuals with evidence of esophagitis, one as determined by histopathology and three as noted on endoscopy.

The WALDO algorithm compares the normalized read counts of 500 kb intervals to intervals on other chromosome arms in the same sample. Its normalization is therefore internal, “within-sample.” The intervals are aggregated across the entire length of the chromosome arm to produce an arm level statistical significance score (Zw). The 39 non-acrocentric Zw values serve as features that are integrated and modeled with a support vector machine (SVM) to provide a summary Global Aneuploidy Score that discriminates between aneuploid and euploid samples. The SVM classifier was trained on 1,334 normal euploid plasma samples and 2,016 in silico aneuploid samples generated from the normal plasma samples. The in silico samples were generated to mimic recurrently altered chromosome arms observed in cancers, including esophageal cancers. In addition to generating arm level statistical significances, the circular binary segmentation algorithm was applied to identify sub-chromosomal focal alterations. Note that the SVM classifier and segmentation algorithm were identical to those used to evaluate previous data on plasma. The only difference in the algorithmic component used in the current study was that the 500 kb clusters used to define aneuploidy in the test sample were generated from seven matched esophageal samples from normal individuals rather than seven matched plasma samples from normal individuals. RealSeqS, WALDO, and the SVM classifier were all performed by investigators blinded to the clinical classification of the associated samples. The software used to generate the scores is available at (https://doi.org/10.5281/zenodo.3656943)

### Post-Hoc Review of Clinical Follow-Up

Although the study was cross sectional in design, limited follow-up data were available and were retrieved following completion of all aneuploidy analysis and classification. Follow-up results were retrieved by an investigator via review of the electronic health and pathology results on biopsies performed during surveillance endoscopies subsequent to the research brush sampling.

### Results

### Patient Characteristics

The cohort consisted of 79 patients in the training set (15 samples from normal gastroesophageal junctions, 19 with NDBE, 15 with HGD, and 30 with EAC) and 268 patients in the validation set (84 samples from normal gastroesophageal junction, 41 with NDBE, 32 with LGD, 28 with HGD, and 83 with EAC). LGD samples were not included in the training set because of known challenges in the reproducibility of expert pathologists in classification of LGD. There were no statistically significant differences between the demographic compositions of the training and validation sets except for the racial makeup among the unaffected controls.

The general demographics of the training and validation sets including race, gender, smoking history, and age are presented in Tables 3, 4 and 5).

### Training Set

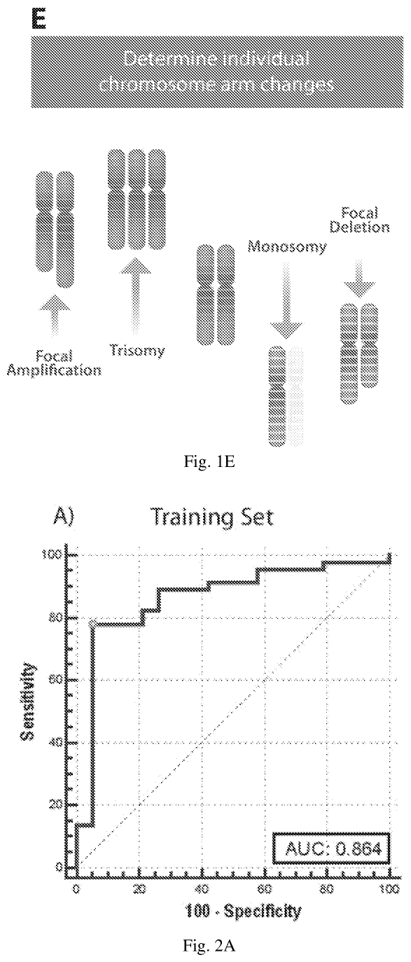

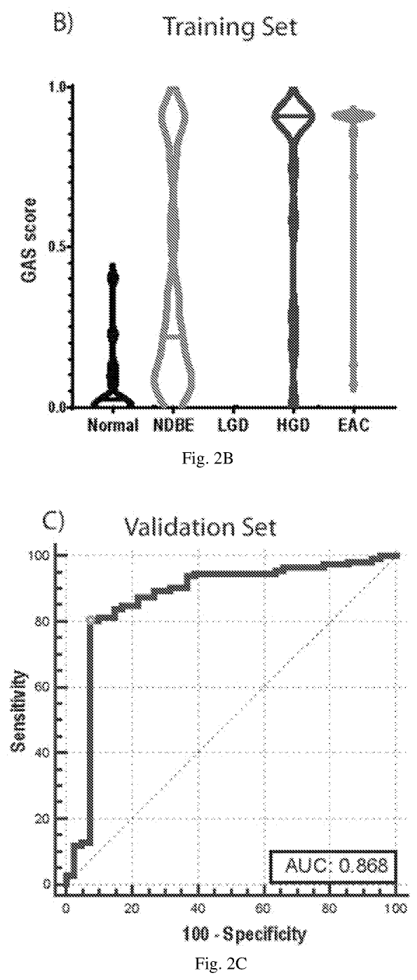

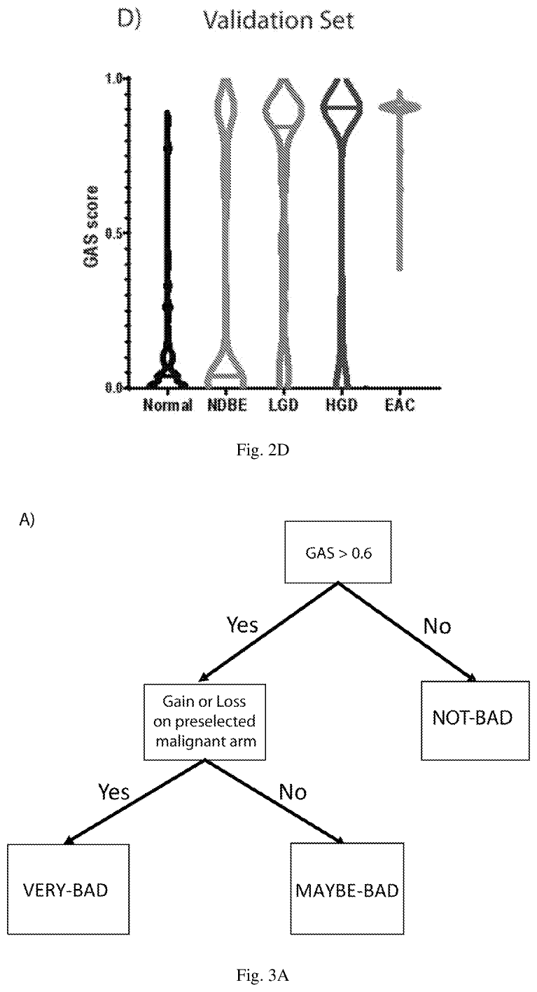

We first evaluated aneuploidy in the Training Set. Zw scores for each of the 39 non-acrocentric chromosome arms in each sample were calculated. These chromosome arm level Zw scores were then integrated into a single Global Aneuploidy Score (GAS) reflecting the number and similarity of the alterations to those commonly observed in cancers (as per Methods). Our major goal was to discriminate patients with advanced lesions that require clinical intervention (HGD and EAC) from those with earlier lesions that do not (i.e., samples with NDBE). Sensitivity was determined by the fraction of patients with advanced lesions who had GAS greater than a given threshold. Analogously, specificity was determined as the corresponding fraction of patients with early lesions having GAS values less than this threshold. The receiver operating characteristic (ROC) curve of sensitivity versus 1-specificity is shown for thresholds ranging from highest to lowest and demonstrates an area under curve (AUC) of 0.864, consistent with GAS providing good discrimination between NDBE versus EAC and HGD (FIG. 2A).

Violin plots of the individual distributions of GAS scores are shown in FIG. 2B. The distributions observed in samples from patients with a normal esophagus, with BE without dysplasia (NDBE), with BE with high-grade dysplasia (HGD), and with carcinoma (EAC) were each significantly different from one another (P<0.01 by the Kolmogorov-Smirnov test).

Interestingly, a clear bimodal distribution of aneuploidy scores was observed in the BE patients without dysplasia (NDBE). One mode was centered at a GAS of 0.2 and the other at a GAS of 0.85. This suggested that a biologically appropriate threshold for a positive GAS should distinguish between these two groups of patients with NDBE. From inspection of the violin plots, a GAS value of 0.6 was chosen for this threshold. At the threshold of 0.6, GAS identified the patient group with advanced lesions of HGD or EAC at a sensitivity of 86.7% and showed specificity for not detecting patients with NDBE of 73.7% (Table 6). We considered that for clinical applications, the medical consequence of missing an advanced lesion (false negatives) was higher than the cost of false positives, and accordingly decided to employ this threshold of 0.6 for all subsequent studies. Hereinafter, samples with a GAS>0.6 are referred to as “aneuploid” and those with GAS≤0.6 as “non-aneuploid”. Note that the non-aneuploid designation is relative; some of the samples with GAS≤0.6 had a small number of chromosome arm alterations, while all of the samples with GAS>0.6 had a larger number of chromosome alterations (Dataset 1).

Identifying Chromosome Changes that Distinguish Histologic Progression of BE

We next characterized which specific chromosome arm changes accompanied progression of BE to EAC, reasoning that emphasizing these specific chromosome alterations might further improve accuracy of the GAS for discriminating histologic progression of disease. To minimize overfitting, we restricted our investigation to the samples that were aneuploid (as defined by a GAS>0.6) in the Training Set.

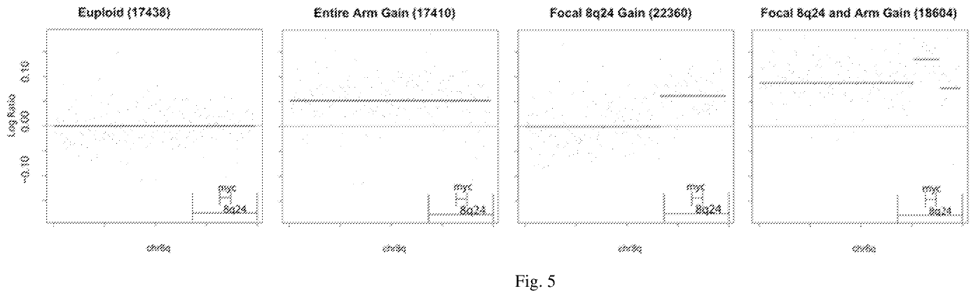

There were five aneuploid BE samples and 28 aneuploid EAC samples in the Training Set (Tables 3 and 6). As there was a low number of samples and large number of possible chromosome arms involved in progression (39 possible gains and 39 possible losses), we sought to determine a minimal set of alterations enabling EAC discrimination. From careful manual inspection of the Training Set data (Table 7), informed by previous studies of mutations and copy number alterations in cancer, we selected a panel of 5 candidate chromosome arms for this purpose: 1q gain, 9p loss, 12p gain, 17p loss, and 20q gain. All five of these arms had been previously implicated in cancer: 1q and 20q gains are very commonly found in cancers; 9p contains tumor suppressor p16; 12p contains oncogene KRAS; and 17p contains tumor suppressor TP53. Further inspection of the Training Set data highlighted focal amplifications surrounding 8q24 as present in 50% of aneuploid EAC and as absent in the 5 aneuploid NBDE (Representative Plots in FIG. 5). The 8q24 region contains the driver gene CMYC and has been reported as one of the most common focal amplifications in EAC; we therefore also incorporated focal amplifications of 8q24 into our panel of alterations used for classification. The presence of any of these six specific chromosomal alterations distinguished all but one the five aneuploid NDBE samples from the 28 aneuploid EAC samples, with most EACs having at least two of these specific chromosomal changes (FIG. 3B).

Consideration of other chromosome alterations did not further improve the accuracy of distinguishing EAC from NDBE in the training set, satisfying our aim of developing a minimal set of alterations for discriminating EAC from NDBE.

Based on the GAS score and this panel of six specific chromosomal alterations, we developed a simple decision tree classifier, termed BAD (Barrett's Aneuploidy Decision), for distinguishing stages of BE progression (FIG. 3A). BAD sorted samples into three categories. Not-BAD cases had GAS≤0.6, indicating relative non-aneuploidy. Maybe-BAD cases had GAS>0.6 but none of the six specific chromosome alterations, possibly indicating a greater potential risk of progression. Very-BAD cases had GAS>0.6 and losses of 9p or 20q, gains of 1q, 12p, or 20q, or a focal gain of 8q24 (FIG. 3A, 3B, Table 7, and DataSets 1 and 2). The BAD classification system, which used both specific chromosome changes plus GAS scores, outperformed GAS scores alone. In particular, compared to the aneuploidy classification of GAS, the Very-BAD classification markedly improved both the specificity for rejecting NDBE and the positive predictive value (PPV) for identifying HGD plus EAC cases. Moreover, compared to aneuploidy alone, the Very-BAD classification produced only minimal decreases in the sensitivity for detecting HGD or EAC or in the negative predictive value (FIG. 3B, Table 6).

Validation Set

The Validation Set provided an opportunity to independently assess the sensitivity and specificity of the BAD classifier. Note that the patients recruited for the Validation Set were entirely distinct from those in the Training Set, and that the GAS and BAD assignments of validation cases were performed by investigators blinded to the clinical status of the samples. The first important observation in the Validation Set was that the ROC curve for the GAS component of the BAD classifier was strikingly similar to that in the Training Set (FIG. 2A versus 2C). For example, the AUCs were 0.86 and 0.87 in the Training and Validation Sets, respectively. Violin plots of the GAS for each of group of patients in the Validation Set are shown in FIG. 2D, and closely resemble those obtained in the Training Set (FIG. 2B). Importantly, samples from the patients with NDBE in the Validation Set exhibited the same bimodal distribution as observed in the Training Set, with the GAS threshold of 0.60 again cleanly separating Validation Set NDBE into two populations, 36.6% as aneuploid and 63.4% as non-aneuploid (FIG. 2D and Table 6).

Moreover, when the BAD classifier was applied to the Validation Set, it again was more accurate than GAS alone. For example, it more accurately discriminated the majority of samples with histologic evidence of disease progression from those with NDBE. Specifically, 96.4% of EAC and 67.9% of HGD were classified as Very-BAD (FIG. 3C, Table 1, Tables 4, 6, and DataSet 3). In contrast, only 7.3% of NDBE were classified as Very-BAD (FIG. 3C, Table 1, Table 6). Moreover, intramucosal cancer (IMCA), the earliest stage of EAC, largely curable with endoscopic techniques, was detected as Very-BAD in three of three cases (Table 3). Furthermore, the sensitivity for detecting HGD as Very-BAD increased to 72.7% when considering only cases with more than 1 mm of HGD present in the diagnostic biopsy (i.e., cases with more than “focal” HGD extent, N=22) (Table 4).

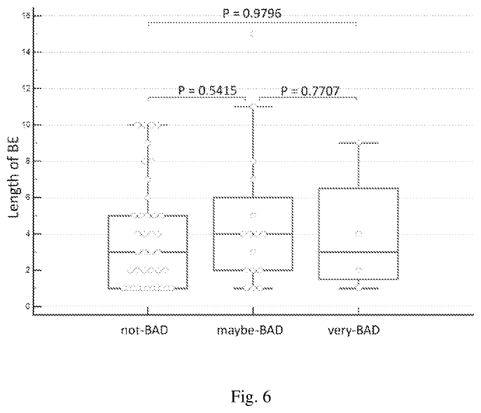

Among Validation Set NDBE cases, 7.3% were classified as Very-BAD, 29.3% as Maybe-BAD, and 63.4% as Not-BAD (Table 1). Thus, the Validation Set resembled the Training Set in that approximately one third of the patients with NDBE harbored aneuploid cells, but the BAD classifier could distinguish nearly all these aneuploid NDBE from the aneuploid HGD and EAC cases (Table 1). Among NDBE patients studied, acquisition of either Very-BAD or Maybe-BAD status showed no significant relation to BE length, with mean BE segment lengths of 3.9 cm for Not-BAD NDBE (95% confidence interval 3.0 to 4.9 cm), versus 4.0 cm for Very-BAD NDBE (95% confidence interval 0 to 9.7 cm), and 4.6 cm for Maybe-BAD NDBE (95% confidence interval 2.5 to 6.7) (P=0.787 by one-way ANOVA) (FIG. 6). Additionally, there was no significant difference between performance of the BAD classifier in the total Validation Set versus among the 76% subset of cases for which central pathology review was available (Table 8).

None (0%) of 84 Validation Set normal controls were scored as Very-Bad, three (3.6%) were scored as Maybe-BAD, and the remaining 96.4% were scored as Not-Bad (Table 1 and Table 4).

The Validation Set included samples from BE patients with LGD. These samples also demonstrated a bimodal distribution, with one group having high GAS scores and the other low GAS scores (FIG. 2). Among these LGD cases, 50.0% were classified as Very-BAD, 21.9% as Maybe-BAD, and 28.1% as Not-BAD (Table 1). This difficulty in classifying the status of LGD cases with respect to aneuploidy mirrors the well-known difficulties in histopathological classification of LGD. As noted above, some experienced pathologists classify LGD as NDBE and others classify the same sample as HGD.

In the Validation Set, as in the Training Set, the BAD classification system outperformed GAS scores alone. Compared to the aneuploidy classification of GAS, the Very-BAD classification again markedly improved both the specificity for rejecting NDBE and the positive predictive value (PPV) for identifying HGD plus EAC validation cases. And again, compared to aneuploidy alone, the Very-BAD classification produced only minimal decreases in the sensitivity for detecting Validation Set HGD or EAC or in the negative predictive value (FIG. 3C, Table 6).

### Clinical Review for Disease Progression

Following analysis of the validation set, an expert central reviewer (AC), who was blinded to the RealSeqS results, reviewed the medical records of all NDBE cases in both the Training and Validation Sets for any evidence of disease progression subsequent to study entry. Four of the 60 NDBE cases in the total cohort were Very-BAD, and 2 of these 4 were found to have progressed within 36 months of study entry, one to HGD and one to EAC. In contrast, no instances of histopathologic progression were identified during equivalent follow-up of the other 56 NDBE (nominal P<0.004), including 40 Not-BAD and 16 Maybe-BAD cases. Progression to HGD within three years was also identified in two individuals with LGD, despite both having undergone disease ablation. At study entry one of these individuals was classified as Very-BAD and the other as Maybe-BAD. No progression was identified in the 10 Not-BAD LGD cases, including 5 individuals who did not undergo disease ablation. In total, of 4 individuals who developed disease progression within 36 months of study entry, 3 had antecedent brushings testing as Very-BAD.

### Expanded Analysis of Chromosome Arm Changes During Progression

We designed only one classifier on the basis of the Training Set data so that we could rigorously evaluate the Validation Set in a statistically sound fashion. Had we designed several classifiers, then we would have had to correct performance in the Validation Set for multiple hypothesis testing. However, after evaluating the Validation Set with our single BAD classifier, we thought it of interest to evaluate how alternative classifier panels might be constructed for future studies.

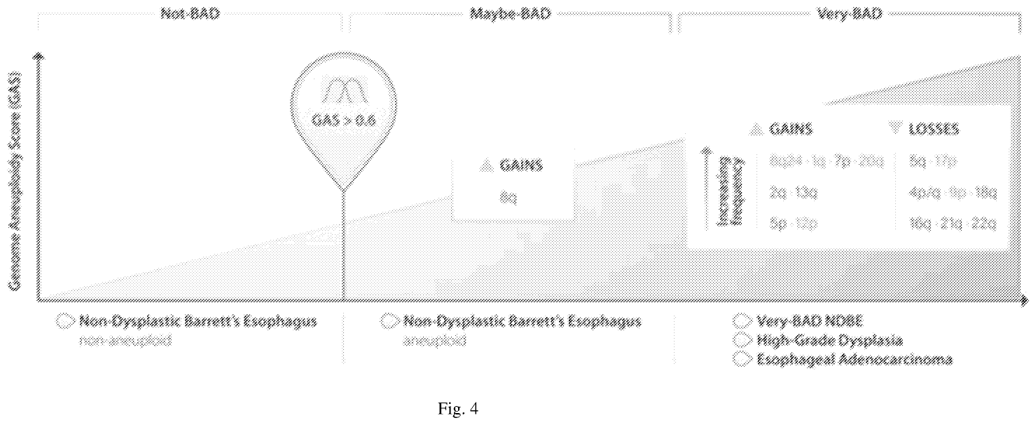

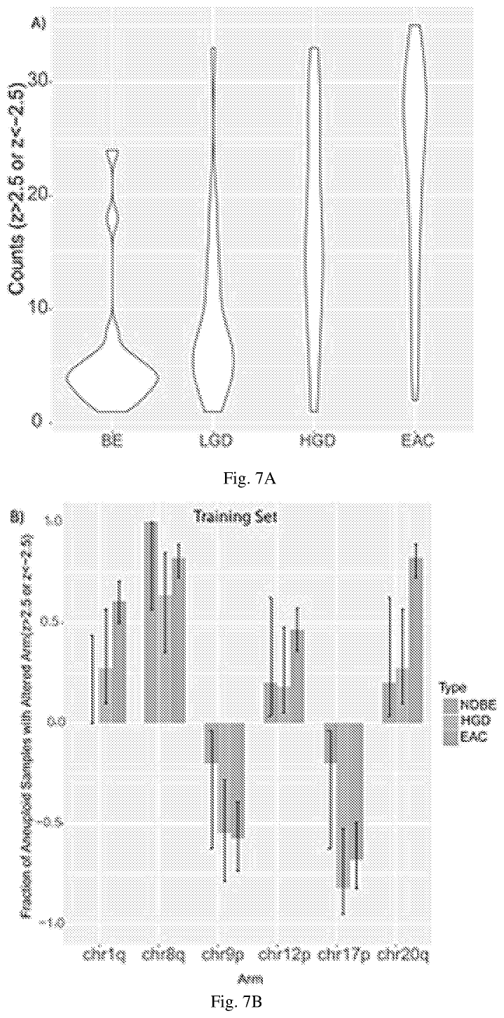

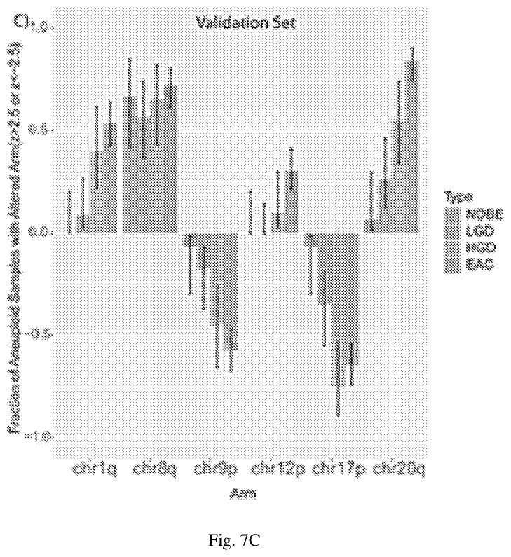

To explore this question, we conducted a secondary analysis that used all aneuploid samples from the combined Training and Validation Sets to characterize alterations during progression of each of the individual chromosome arms. We observed that the total number of chromosome arms lost or gained steadily increased during disease progression. The average number of altered chromosome arms was 6 for aneuploid NDBE, 10 for aneuploid LGD, 17 for aneuploid HGD, and 22 for aneuploid EAC (FIG. 7A). The chromosome arm abnormality counts in the four stages of disease were each significantly different from one another (P value<0.05 by the Student's t-test). Inspection of the individual chromosomes showed that 16 specific chromosome arm gains and 14 specific chromosome arm losses had a difference of greater than 23% in EAC compared to aneuploid NDBE, and each of these differences was statistically significant (P value<0.05 by the Binomial Proportions test) (Table 7). All five chromosome arms selected from Training Set data for inclusion in the BAD decision tree were among those. The most prominent chromosome arm changes observed with progression are depicted in FIG. 4, and, as expected, included the five chromosome arms selected from the Training Set data for inclusion in the BAD decision tree (FIGS. 3B, 3C, 7B, 7C, and Table 9).

### Chromosome 8q Gains are Found in Aneuploid NDBE and Evolve During Progression to EAC

We next investigated what chromosome alterations accounted for the aneuploidy detected in 37% of NDBE. Examination of these aneuploid NDBE cases revealed that chromosome alterations were not entirely random and were typified by gains of 8q. Of the 20 aneuploid NDBE samples in the complete study cohort, 15 (75%) had an 8q gain. This was by far the most common alteration in the aneuploid NDBE samples and is one of the most common chromosome alterations in cancer in general. Furthermore, the fraction of aneuploid BE samples with an 8q gain was not significantly different in patients with NDBE, LGD, HGD, or EAC (FIGS. 7B and 7C). Chromosome 8q in NDBE was different from all other chromosome arms, for which the frequency of alteration increased concomitantly with disease progression (FIG. 7B, 7C, and Table 9).

Overall, of 60 patients with NDBE, 18 cases (15 with GAS>0.6 and three cases with GAS<0.6) harbored a gain of 8q (DataSet 1). In 100% of these 18 NDBE instances, the entire 8q chromosome arm was gained (FIG. 5). Moreover, a gain of the entire chromosome 8q was detected as the sole chromosomal change in five (8.3%) of the 60 NDBE cases evaluated. In EACs, this pattern (“only 8q gain”) was seen in only two (1.8%) of 113 cases (nominal P value=0.05). This suggested that gain of the entire chromosome 8q arm as a sole chromosomal alteration is a biomarker of early aneuploidy development in BE. As implied above, none of the brushings from the four patients who progressed to HGD or EAC, starting from BE or LGD, showed this “only 8q gain” pattern.

Similar to NDBE, 82 of 110 (75%) EAC cases also demonstrated an 8q gain. However, in contrast to NDBE, in only 36 of these 82 EAC cases (44%) was all of 8q gained (FIG. 5). In the remaining 46 EAC cases (56%), 8q gains were all sub-chromosomal, in every case encompassing 8q24 (nominal P value=0.0004) (examples depicted in FIG. 5) (FIGS. 3B and 3C, DataSets 2 and 3). Intriguingly, the pattern of 8q gains in LGD and HGD more closely resembled that of NDBE than EAC (FIGS. 3B and 3C. DataSets 2 and 3). In LGD, 13 cases demonstrated 8q gain, and 11 of these 13 cases showed gains of the entire 8q. Similarly, in HGD, 19 cases demonstrated 8q gains, and 17 of these 19 cases showed gains of all of 8q. Thus, alterations of 8q may be initiated in NDBE, beginning as whole arm gains, and may evolve in EAC to focus on 8q24, accompanied by stepwise increases in the copy number of CMYC.

### Reproducibility

Duplicate esophageal brushings obtained at the same clinical session were available from 22 participants in this study. These included eight unaffected controls, four cases of LGD, five cases of HGD, and five cases of EAC. In 21 of the 22 instances (95%), the determinations from the independent duplicate brushings were fully concordant, both with respect to their GAS determination of aneuploidy as well as their further classification as Not-BAD, Maybe-BAD, or Very-BAD (Table 2 and DataSet 4). In the other case, an LGD, the duplicate brushings were discordant, with one classified as Not-BAD and the other as Very-BAD (Table 2, Supplementary Table 10 and DataSet 4).

Multiple related observations and insights were garnered from this study. First, the combination of esophageal brushings with RealSeqS analysis provides a practical and sensitive method for detecting chromosome arm alterations in BE patients. Second, aneuploidy typifies EAC and HGD, but also can be identified in a small proportion of NDBE and a larger proportion of LGD patients. Third, alterations of specific chromosome arms containing well-known driver genes are found commonly in late stage but rarely in early stage disease. Fourth, there is a special role for chromosome 8q in BE progression, with gains of the whole of 8q appearing to be an early event, present in 75% of aneuploid NDBE, and with selective focused gains on 8q24 often found in EAC. And fifth and most importantly, these chromosomal alterations can be used to design a molecular classifier called BAD, and the BAD classification of DNA from esophageal brushings is highly correlated with histopathologic classification of the same patients (visually depicted in FIG. 4).

The power of the approach described here comes from the juxtaposition of the two major components described above. Brushings can sample a much more extensive region of the esophagus than conventional biopsies, even when multiple biopsies are performed. But this extensive and convenient sampling comes with a cost: the aneuploidy present in the dysplastic cells within any individual lesion is diluted with non-dysplastic cells from the remaining esophagus. Fortunately, RealSeqS technology is well-suited for the detection of a relatively small fraction of aneuploid cells admixed with a much larger number of non-aneuploid cells.

The chromosomal alterations identified by RealSeqS analysis of esophageal brushings are consistent with previous studies of EAC tissues that employed more traditional genetic methods and/or fluorescence in situ hybridization (FISH) analysis.

Shallow whole genome sequencing (WGS), in principle, should also be able to detect aneuploidy in esophageal brushings. Shallow whole genome sequencing has been productively used for many purposes, particularly for non-invasive prenatal testing. RealSeqS has some advantages over WGS. First, it requires only a tiny amount of input DNA and is exceedingly simple to perform, as it employs PCR with a single pair of primers to prepare samples for massively parallel sequencing. WGS requires several steps, including shearing and library preparation, prior to sequencing. Additionally, at the same sequencing depth, whole genome sequencing is not as sensitive as RealSeqS for detecting relatively small fractions of aneuploid cells, particularly when aneuploidy is of small chromosomes. On the other hand, an advantage of WGS is that it can reveal information about chromosome regions that are not queried in RealSeqS because the latter evaluates only ˜350,000 repetitive elements rather than the entire genome.

Along with this study, other studies have also advanced alternative technologies to improve the identification of BE patients at risk of progressing to EAC. These include: the combination of enhanced image analysis together with molecular markers to predict progression risk from individual BE biopsies and the combination of extensive and deep tissue brushing of BE with enhanced image analysis to increase the identification of dysplasia. Future studies will be required to further evaluate the role of these different technologies in the early identification of high risk BE patients and to provide direct comparison of their performance to the technology we advance here.

On the other hand, a particularly intriguing observation of the current study was that patients with relatively early phase BE are heterogeneous with respect to chromosomal changes, with a minority of NDBE (7%) cases and half of LGD cases classified as Very-BAD. This suggests the possibility that it is the Very-BAD subset of patients with NDBE or LGD that are at greatest risk for progression. Indeed, three of the four individuals who were later shown to progress to HGD or EAC were classified as Very-BAD in our study.

The observations described above, in combination with prior studies establishing the association of aneuploidy with BE progression, suggest several hypothetical clinical implications. First, RealSeqS analysis of esophageal brushings may provide a technique that could be combined with the current Seattle protocol of multiple random biopsies to augment the effectiveness of BE surveillance for detecting early progression to dysplasia or cancer. This combination could minimize the development of interval cancers between BE surveillance sessions, which is at present a widely-recognized challenge. Second, the NDBE cases classified as Very-BAD may benefit from intensified surveillance, while the Not-BAD cases may require less surveillance. Similarly, the LGD cases classified as Very-BAD may benefit from ablation therapies, whereas Not-BAD cases could potentially be followed with continued endoscopic surveillance. If confirmed, this would potentially enable molecular classification to add to current morphologic criteria for risk stratification.

In overview, the current study demonstrates that the combination of esophageal brushing with RealSeqS can molecularly discriminate different stages during BE progression and can detect the majority of prevalent histologically advanced lesions.

TABLE 1
Performance of the RealSeqS BAD Classifier

| | Training Set | | | Validation Set | | | Training Plus Validation Sets. | | |
|---|---|---|---|---|---|---|---|---|---|
| | N = 79 | | | N = 268 | | | N = 347 | | |
| | VERY- | MAYBE- | # | VERY- | MAYBE- | # | VERY- | MAYBE- | # |
| | BAD | BAD | Patients | BAD | BAD | Patients | BAD | BAD | Patients |
| GEJ from | 0.0% | 0.0% | 15 | 0.0% | 3.6% | 84 | 0.0% | 3.0% | 99 |
| Unaffected | | | | | | | | | |
| controls | | | | | | | | | |
| NDBE | 5.3% | 21.0% | 19 | 7.3% | 29.3% | 41 | 6.7% | 26.7% | 60 |
| LGD | | | 0 | 50.0% | 21.9% | 32 | 50.0% | 21.9% | 32 |
| HGD | 60.0% | 13.3% | 15 | 67.9% | 3.6% | 28 | 65.1% | 7.0% | 43 |
| EAC | 90.0% | 3.3% | 30 | 96.4% | 2.4% | 83 | 94.7% | 2.7% | 113 |
| EAC + HGD | 80.0% | 6.7% | 45 | 89.2% | 2.7% | 111 | 86.5% | 3.8% | 156 |
| combined** | | | | | | | | | |

**Not an independent category, but the sum of the above HGD and EAC groups.

TABLE 2
Concordance between duplicate brushes

| Diagnosis | Concordant positive | Concordant negative | Discordant |
|---|---|---|---|
| Unaffected | | 8 | |
| Control | | | |
| LGD | 2 | 1 | 1 |
| HGD | 4 | 1 | |
| EAC | 5 | | |
| Totals | 11 | 10 | 1 |

Concordance status for both GAS status and BAD classifications was identical in all samples.

TABLE 3
Training Set Demographics and Ploidy Results

| Patient | Patient | | | Age | | | Former or | | BAD | | | |
|---|---|---|---|---|---|---|---|---|---|---|---|---|
| Study | custom | | Brushing | [>90 = | | | Current | Ploidy | tree | Focal | | |
| Number | IDs | Diagnosis | ID | 90] | Race | Gender | Smoker | Result | call | HGD? | Stage | IMCA |
| 3 | AA-0009- | Normal | 17560 | 54 | White | Male | Yes | Euploid | not- | | — | — |
| | 01-0010 | | | | | | | | BAD | | | |
| 6 | AA-0168- | Normal | 22401 | 53 | White | Female | Unknown | Euploid | not- | | — | — |
| | 01-0001 | | | | | | | | BAD | | | |
| 7 | AA-1197- | Normal | 17618 | 57 | White | Male | Yes | Euploid | not- | | — | — |
| | 01-0003 | | | | | | | | BAD | | | |
| 8 | AA-1383- | Normal | 17438 | 51 | White | Male | Yes | Euploid | not- | | — | — |
| | 01-0001 | | | | | | | | BAD | | | |
| 9 | AA-1420- | Normal | 17476 | 56 | White | Female | No | Euploid | not- | | — | — |
| | 01-0001 | | | | | | | | BAD | | | |
| 31 | AA-1566- | Normal | 22421 | 75 | White | Female | No | Euploid | not- | | — | — |
| | 01-0001 | | | | | | | | BAD | | | |
| 33 | AA-1575- | Normal | 18570 | 88 | White | Male | Yes | Euploid | not- | | — | — |
| | 01-0001 | | | | | | | | BAD | | | |
| 34 | AA-1580- | Normal | 18548 | 67 | White | Male | No | Euploid | not- | | — | — |
| | 01-0001 | | | | | | | | BAD | | | |
| 39 | AA-1697- | Normal | 22414 | 42 | White | Male | Unknown | Euploid | not- | | — | — |
| | 01-0001 | | | | | | | | BAD | | | |
| 40 | AA-1699- | Normal | 22415 | 65 | White | Male | Unknown | Euploid | not- | | — | — |
| | 01-0001 | | | | | | | | BAD | | | |
| 41 | AA-1711- | Normal | 22418 | 42 | White | Male | No | Euploid | not- | | — | — |
| | 01-0001 | | | | | | | | BAD | | | |
| 42 | AA-1713- | Normal | 22419 | 84 | White | Female | Unknown | Euploid | not- | | — | — |
| | 01-0001 | | | | | | | | BAD | | | |
| 50 | AA-1783- | Normal | 22407 | 61 | White | Female | No | Euploid | not- | | — | — |
| | 01-0001 | | | | | | | | BAD | | | |
| 54 | AA-1791- | Normal | 22422 | 69 | White | Male | No | Euploid | not- | | — | — |
| | 01-0001 | | | | | | | | BAD | | | |
| 56 | AA-1795- | Normal | 22425 | 64 | White | Female | No | Euploid | not- | | — | — |
| | 01-0001 | | | | | | | | BAD | | | |
| 100 | 16006 | BE | 22324 | 62 | White | Male | Yes | Euploid | not- | | — | — |
| | | | | | | | | | BAD | | | |
| 102 | AA-0006- | BE | 17446 | 68 | White | Female | Yes | Euploid | not- | | — | — |
| | 01-0007 | | | | | | | | BAD | | | |
| 104 | AA-0747- | BE | 17512 | 68 | White | Male | No | Euploid | not- | | — | — |
| | 01-0001 | | | | | | | | BAD | | | |
| 105 | AA-0773- | BE | 17632 | 50 | White | Female | Yes | Aneuploid | maybe- | | — | — |
| | 01-0001 | | | | | | | | BAD | | | |
| 106 | AA-0865- | BE | 22309 | 73 | White | Male | Yes | Aneuploid | maybe- | | — | — |
| | 01-0002 | | | | | | | | BAD | | | |
| 107 | AA-1081- | BE | 22305 | 74 | White | Female | No | Euploid | not- | | — | — |
| | 01-0001 | | | | | | | | BAD | | | |
| 111 | AA-1404- | SSBE | 22310 | 84 | White | Female | No | Aneuploid | maybe- | | — | — |
| | 02-0002 | | | | | | | | BAD | | | |
| 113 | AA-1549- | SSBE | 18468 | 28 | White | Male | Yes | Euploid | not- | | — | — |
| | 01-0001 | | | | | | | | BAD | | | |
| 115 | AA-1729- | SSBE | 22254 | 52 | White | Male | No | Euploid | not- | | — | — |
| | 01-0001 | | | | | | | | BAD | | | |
| 117 | AA-1751- | SSBE | 22257 | 62 | White | Male | Yes | Aneuploid | very- | | — | — |
| | 01-0001 | | | | | | | | BAD | | | |
| 145 | BB-0195- | SSBE | 18566 | 57 | White | Male | Yes | Euploid | not- | | — | — |
| | 01-0001 | | | | | | | | BAD | | | |
| 149 | KK-0329- | SSBE | 22275 | 40 | White | Male | Yes | Euploid | not- | | — | — |
| | 01-0001 | | | | | | | | BAD | | | |
| 150 | KK-0334- | SSBE | 22278 | 66 | White | Male | Yes | Euploid | not- | | — | — |
| | 01-0001 | | | | | | | | BAD | | | |
| 151 | MM-0057- | BE | 22313 | 56 | White | Male | No | Euploid | not- | | — | — |
| | 01-0001 | | | | | | | | BAD | | | |
| 152 | MM-0067- | BE | 22315 | 73 | White | Female | Yes | Euploid | not- | | — | — |
| | 01-0001 | | | | | | | | BAD | | | |
| 153 | MM-0072- | SSBE | 22316 | 52 | White | Female | Yes | Aneuploid | maybe- | | — | — |
| | 01-0001 | | | | | | | | BAD | | | |
| 154 | MM-0075- | BE | 22317 | 69 | White | Female | Yes | Euploid | not- | | — | — |
| | 01-0001 | | | | | | | | BAD | | | |
| 155 | MM-0079- | BE | 22318 | 52 | White | Male | Yes | Euploid | not- | | — | — |
| | 01-0001 | | | | | | | | BAD | | | |
| 156 | MM-0081- | BE | 22319 | 75 | White | Male | No | Euploid | not- | | — | — |
| | 01-0001 | | | | | | | | BAD | | | |
| 192 | 16002 | HGD | 22361 | 75 | White | Male | No | Euploid | not- | | — | — |
| | | | | | | | | | BAD | | | |
| 194 | AA-1326- | HGD | 17410 | 63 | White | Male | No | Aneuploid | very- | | — | — |
| | 01-0001 | | | | | | | | BAD | | | |
| 195 | AA-1347- | HGD | 17404 | 59 | White | Male | Yes | Aneuploid | very- | | — | — |
| | 01-0001 | | | | | | | | BAD | | | |
| 196 | AA-1476- | HGD | 18604 | 75 | White | Male | Yes | Aneuploid | very- | | — | — |
| | 01-0001 | | | | | | | | BAD | | | |
| 198 | AA-1600- | HGD | 18526 | 72 | White | Female | Yes | Euploid | not- | | — | — |
| | 01-0001 | | | | | | | | BAD | | | |
| 199 | AA-1629- | HGD | 22348 | 63 | White | Male | No | Aneuploid | very- | | — | — |
| | 01-0001 | | | | | | | | BAD | | | |
| 200 | AA-1703- | HGD | 22349 | 84 | White | Male | Yes | Aneuploid | maybe- | | — | — |
| | 01-0001 | | | | | | | | BAD | | | |
| 201 | AA-1719- | HGD | 22350 | 56 | White | Male | No | Euploid | not- | | — | — |
| | 01-0001 | | | | | | | | BAD | | | |
| 202 | AA-1739- | HGD | 22351 | 65 | White | Male | Yes | Euploid | not- | focal | — | — |
| | 01-0001 | | | | | | | | BAD | | | |
| 203 | AA-1746- | HGD | 22353 | 62 | White | Female | Yes | Aneuploid | maybe- | | — | — |
| | 01-0001 | | | | | | | | BAD | | | |
| 204 | AA-1749- | HGD | 22354 | 81 | White | Female | Yes | Aneuploid | very- | | — | — |
| | 01-0001 | | | | | | | | BAD | | | |
| 205 | 1-0001 AKA | HGD | 22355 | 68 | Black | Male | Yes | Aneuploid | very- | | — | — |
| | BB-006 | | | | | | | | BAD | | | |
| 206 | AA-1757- | HGD | 22356 | 61 | White | Female | No | Aneuploid | very- | | — | — |
| | 01-0001 | | | | | | | | BAD | | | |
| 233 | KK-0373- | HGD | 22280 | 69 | White | Male | No | Aneuploid | very- | | — | — |
| | 01-0001 | | | | | | | | BAD | | | |
| 234 | MM-0078- | HGD | 22358 | 73 | White | Male | No | Aneuploid | very- | | — | — |
| | 01-0000 | | | | | | | | BAD | | | |
| 236 | 15020 | EAC | 22397 | 69 | White | Male | No | Aneuploid | very- | | I | IMCA |
| | | | | | | | | | BAD | | | |
| 238 | 16004 | EAC | 22395 | 83 | White | Male | Yes | Euploid | not- | | III | No |
| | | | | | | | | | BAD | | | |
| 240 | 16007 | EAC | 22393 | 47 | White | Female | No | Euploid | not- | | I | No |
| | | | | | | | | | BAD | | | |
| 241 | 16008 | EAC | 22392 | 73 | White | Male | No | Aneuploid | very- | | III | No |
| | | | | | | | | | BAD | | | |
| 242 | KK-0336- | EAC | 22383 | 69 | White | Male | Yes | Aneuploid | very- | | IV | No |
| | 01-0001 | | | | | | | | BAD | | | |
| 247 | 93B | EAC | 22388 | 58 | White | Male | No | Aneuploid | very- | | III | No |
| | | | | | | | | | BAD | | | |
| 249 | 98B | EAC | 22390 | 66 | White | Male | No | Aneuploid | very- | | II | No |
| | | | | | | | | | BAD | | | |
| 250 | AA-0002- | EAC | 22362 | 87 | White | Male | No | Aneuploid | very- | | I | No |
| | 01-0002 | | | | | | | | BAD | | | |
| 253 | AA-1360- | EAC | 17424 | 79 | White | Female | No | Aneuploid | very- | | III | No |
| | 01-0001 | | | | | | | | BAD | | | |
| 257 | AA-1388- | EAC | 17444 | 77 | White | Male | Yes | Aneuploid | very- | | i | No |
| | 01-0001 | | | | | | | | BAD | | | |
| 258 | AA-1405- | EAC | 17458 | 79 | White | Male | No | Aneuploid | very- | | III | No |
| | 01-0001 | | | | | | | | BAD | | | |
| 261 | AA-1414- | EAC | 17466 | 73 | White | Male | Yes | Aneuploid | very- | | 0 | No |
| | 01-0001 | | | | | | | | BAD | | | |
| 262 | AA-1426- | EAC | 17482 | 67 | White | Male | Yes | Aneuploid | very- | | III | No |
| | 01-0001 | | | | | | | | BAD | | | |
| 266 | AA-1452- | EAC | 17526 | 64 | White | Male | No | Aneuploid | very- | | IV | No |
| | 01-0001 | | | | | | | | BAD | | | |
| 267 | AA-1457- | EAC | 17532 | 64 | White | Male | Yes | Aneuploid | very- | | III | No |
| | 01-0001 | | | | | | | | BAD | | | |
| 270 | AA-1495- | EAC | 17602 | 59 | White | Male | Yes | Aneuploid | very- | | II | No |
| | 01-0001 | | | | | | | | BAD | | | |
| 275 | AA-1523- | EAC | 18504 | 49 | White | Male | Yes | Aneuploid | very- | | III | No |
| | 01-0001 | | | | | | | | BAD | | | |
| 276 | AA-1534- | EAC | 18512 | 75 | White | Male | Yes | Aneuploid | very- | | III | No |
| | 01-0001 | | | | | | | | BAD | | | |
| 283 | AA-1695- | EAC | 22363 | 89 | White | Female | No | Aneuploid | maybe- | | III | No |
| | 01-0001 | | | | | | | | BAD | | | |
| 285 | AA-1707- | EAC | 22365 | 58 | White | Male | Yes | Aneuploid | very- | | I | No |
| | 01-0001 | | | | | | | | BAD | | | |
| 286 | AA-1717- | EAC | 22366 | 57 | White | Male | Yes | Aneuploid | very- | | IV | No |
| | 01-0001 | | | | | | | | BAD | | | |
| 287 | AA-1718- | EAC | 22367 | 75 | White | Male | Yes | Aneuploid | very- | | III | No |
| | 01-0001 | | | | | | | | BAD | | | |
| 289 | AA-1725- | EAC | 22369 | 69 | White | Male | Yes | Aneuploid | very- | | III | No |
| | 01-0001 | | | | | | | | BAD | | | |
| 290 | AA-1734- | EAC | 22370 | 80 | White | Male | No | Aneuploid | very- | | I | No |
| | 01-0001 | | | | | | | | BAD | | | |
| 291 | AA-1735- | EAC | 22371 | 86 | White | Male | Yes | Aneuploid | very- | | III | No |
| | 01-0001 | | | | | | | | BAD | | | |
| 292 | AA-1736- | EAC | 22372 | 80 | White | Male | Yes | Aneuploid | very- | | II | No |
| | 01-0001 | | | | | | | | BAD | | | |
| 294 | AA-1747- | EAC | 22374 | 52 | White | Male | Yes | Aneuploid | very- | | IV | No |
| | 01-0001 | | | | | | | | BAD | | | |
| 295 | AA-1748- | EAC | 22375 | 67 | White | Female | Yes | Aneuploid | very- | | 0 | No |
| | 01-0001 | | | | | | | | BAD | | | |
| 301 | AA-1786- | EAC | 22378 | 65 | White | Male | Yes | Aneuploid | very- | | III | No |
| | 01-0001 | | | | | | | | BAD | | | |
| 346 | MM-0053- | EAC | 22380 | 86 | White | Female | No | Aneuploid | very- | | II | No |
| | 01-0001 | | | | | | | | BAD | | | |

TABLE 4
Validation Set Demographics and Ploidy Results

| | | | | | | | Former | | | Concordant | | | |
|---|---|---|---|---|---|---|---|---|---|---|---|---|---|
| Patient | Patient | | ID | Age | | | or | | BAD | or | | | |
| Study | custom | | Brushing | [>90 = | | | Current | Ploidy | tree | Discordant | Focal | | |
| Number | IDs | Diagnosis | #1 | 90] | Race | Gender | Smoker | Result* | call | Pair | HGD? | Stage | IMCA |

| | | | | | | | | | | | |
|---|---|---|---|---|---|---|---|---|---|---|---|
| 1 | 15016 | Normal | 22428 | 60 | White | Female | No | Euploid | not-BAD | — | — |
| 2 | AA- | Normal | 24031 | 51 | White | Female | No | Euploid | not-BAD | — | — |
| | 0001- | | | | | | | | | | |
| | 03-0005 | | | | | | | | | | |
| 4 | AA- | Normal | 17582 | 24 | White | Male | Yes | Euploid | not-BAD | — | — |
| | 0009- | | | | | | | | | | |
| | 03-0003 | | | | | | | | | | |
| 5 | AA- | Normal | 24032 | 66 | White | Male | No | Euploid | not-BAD | — | — |
| | 0159- | | | | | | | | | | |
| | 01-0001 | | | | | | | | | | |
| 10 | AA- | Normal | 17478 | 63 | Black | Male | Yes | Euploid | not-BAD | — | — |
| | 1423- | | | | | | | | | | |
| | 01-0001 | | | | | | | | | | |
| 11 | AA- | Normal | 17492 | 51 | White | Male | Yes | Euploid | not-BAD | — | — |
| | 1434- | | | | | | | | | | |
| | 01-0001 | | | | | | | | | | |
| 12 | AA- | Normal | 17494 | 28 | Black | Female | No | Euploid | not-BAD | — | — |
| | 1435- | | | | | | | | | | |
| | 01-0001 | | | | | | | | | | |
| 13 | AA- | Normal | 17506 | 46 | Black | Male | Yes | Euploid | not-BAD | — | — |
| | 1443- | | | | | | | | | | |
| | 01-0001 | | | | | | | | | | |
| 14 | AA- | Normal | 17520 | 63 | Black | Female | Yes | Euploid | not-BAD | — | — |
| | 1448- | | | | | | | | | | |
| | 01-0001 | | | | | | | | | | |
| 15 | AA- | Normal | 17540 | 42 | White | Male | Yes | Euploid | not-BAD | — | — |
| | 1455- | | | | | | | | | | |
| | 01-0001 | | | | | | | | | | |
| 16 | AA- | Normal | 17548 | 74 | White | Female | No | Euploid | not-BAD | — | — |
| | 1464- | | | | | | | | | | |
| | 01-0001 | | | | | | | | | | |
| 17 | AA- | Normal | 17570 | 72 | Black | Female | No | Aneuploid | maybe- | — | — |
| | 1478- | | | | | | | | BAD | | |
| | 01-0001 | | | | | | | | | | |
| 18 | AA- | Normal | 17572 | 44 | White | Female | No | Euploid | not-BAD | — | — |
| | 1480- | | | | | | | | | | |
| | 01-0001 | | | | | | | | | | |
| 19 | AA- | Normal | 17576 | 57 | White | Female | No | Aneuploid | maybe- | — | — |
| | 1482- | | | | | | | | BAD | | |
| | 01-0001 | | | | | | | | | | |
| 20 | AA- | Normal | 17588 | 59 | White | Female | No | Euploid | not-BAD | — | — |
| | 1487- | | | | | | | | | | |
| | 01-0001 | | | | | | | | | | |
| 21 | AA- | Normal | 17610 | 44 | Black | Female | Yes | Euploid | not-BAD | — | — |
| | 1498- | | | | | | | | | | |
| | 01-0001 | | | | | | | | | | |
| 22 | AA- | Normal | 17630 | 50 | Black | Female | Yes | Euploid | not-BAD | — | — |
| | 1508- | | | | | | | | | | |
| | 01-0001 | | | | | | | | | | |
| 23 | AA- | Normal | 17636 | 67 | White | Male | Yes | Euploid | not-BAD | — | — |
| | 1515- | | | | | | | | | | |
| | 01-0001 | | | | | | | | | | |
| 24 | AA- | Normal | 18514 | 50 | White | Female | No | Euploid | not-BAD | — | — |
| | 1535- | | | | | | | | | | |
| | 01-0001 | | | | | | | | | | |
| 25 | AA- | Normal | 18482 | 53 | White | Male | Yes | Euploid | not-BAD | — | — |
| | 1536- | | | | | | | | | | |
| | 01-0001 | | | | | | | | | | |
| 26 | AA- | Normal | 18488 | 50 | White | Male | Yes | Euploid | not-BAD | — | — |
| | 1539- | | | | | | | | | | |
| | 01-0001 | | | | | | | | | | |
| 27 | AA | Normal | 18498 | 55 | White | Female | No | Euploid | not-BAD | — | — |
| | 1544- | | | | | | | | | | |
| | 01-0001 | | | | | | | | | | |
| 28 | AA- | Normal | 18470 | 55 | Black | Female | Yes | Euploid | not-BAD | — | — |
| | 1550- | | | | | | | | | | |
| | 01-0001 | | | | | | | | | | |
| 29 | AA- | Normal | 18588 | 24 | White | Male | No | Euploid | not-BAD | — | — |
| | 1556- | | | | | | | | | | |
| | 01-0001 | | | | | | | | | | |
| 30 | AA- | Normal | 18592 | 28 | Black | Female | Yes | Euploid | not-BAD | — | — |
| | 1558- | | | | | | | | | | |
| | 01-0001 | | | | | | | | | | |
| 32 | AA- | Normal | 18584 | 54 | Black | Female | No | Euploid | not-BAD | — | — |
| | 1574- | | | | | | | | | | |
| | 01-0001 | | | | | | | | | | |
| 35 | AA- | Normal | 18530 | 53 | White | Female | Yes | Euploid | not-BAD | — | — |
| | 1589- | | | | | | | | | | |
| | 01-0001 | | | | | | | | | | |

TABLE 5
Demographics summary of training and validation sets

| | | | African | Other/ | | | Other/ | | | Other/ | (median ± |
|---|---|---|---|---|---|---|---|---|---|---|---|
| | Diagnosis | White | American | unknown | male | female | unknown | Yes | No | unknown | StDev) |
| | Normal | 15 | 0 | 0 | 9 | 6 | 0 | 4 | 7 | 4 | 61 ± 13.5 |
| | BE | 19 | 0 | 0 | 12 | 7 | 0 | 13 | 6 | 0 | 62 ± 13.6 |
| Discovery set | | — | — | — | — | — | — | — | — | — | — |
| | HGD | 14 | 1 | 0 | 11 | 4 | 0 | 8 | 7 | 0 | 68 ± 8.2 |
| | EAC | 30 | 0 | 0 | 25 | 5 | 0 | 18 | 12 | 0 | 69 ± 11.5 |
| | Normal | 56 | 25 | 3 | 37 | 47 | 0 | 36 | 46 | 2 | 55 ± 14.1 |
| | BE | 39 | 0 | 2 | 32 | 9 | 0 | 22 | 17 | 2 | 64 ± 13.5 |
| Validation | LGD | 31 | 0 | 1 | 25 | 7 | 0 | 18 | 12 | 2 | 66.5 ± 11 |
| set | | | | | | | | | | | |
| | HGD | 28 | 0 | 0 | 24 | 4 | 0 | 16 | 11 | 1 | 72.5 ± 11.7 |
| | EAC | 81 | 1 | 1 | 73 | 10 | 0 | 60 | 20 | 3 | 70 ± 11.5 |

Note:
the only statistically significant difference between discovery and validation sets is the racial make-up of the Normal controls (p = 0.0096 by Fisher's exact two-tailed test).
All other comparisons between training and validation cohorts have p-value > 0.05
Smoking history (Number is each category)
Age
Race (Number is each category)
Gender (Number is each category)

TABLE 6
Sensitivity, Specificity, Positive and negative predictive values of the GAS and BAD scoring

| | Training Set | | | Validation Set | | | Training Plus Validation Sets. | | |
|---|---|---|---|---|---|---|---|---|---|
| | N = 79 | | | N = 268 | | | N = 347 | | |
| | GAS | | | GAS | | | GAS | | |
| | (=Very- | | | (very- | | | (very- | | |
| | BAD + | | | BAD + | | | BAD + | | |
| | Maybe- | VERY- | # | maybe- | VERY- | # | maybe- | VERY- | # |
| | BAD) | BAD | Patients | BAD) | BAD | Patients | BAD) | BAD | Patients |
| Specificity GEJ from | 100.0% | 100.0% | 15 | 96.4% | 100.0% | 84 | 97.0% | 100.0% | 99 |
| Unaffected controls | | | | | | | | | |
| Specificity NDBE | 73.7% | 94.7% | 19 | 63.4% | 92.7% | 41 | 66.7% | 93.3% | 60 |
| Sensitivity LGD | | | 0 | 71.9% | 50.0% | 32 | 71.9% | 50.0% | 32 |
| Sensitivity HGD | 73.3% | 60.0% | 15 | 71.4% | 67.9% | 28 | 72.1% | 65.1% | 43 |
| Sensitivity EAC | 93.3% | 90.0% | 30 | 98.8% | 96.4% | 83 | 97.3% | 94.7% | 113 |
| Sensitivity | 86.7% | 80.0% | 45 | 91.9% | 89.2% | 111 | 90.4% | 86.5% | 156 |
| EAC + HGD | | | | | | | | | |
| combined* | | | | | | | | | |
| PPV (cases LGD- | 88.6% | 97.3% | 79 | 89.3% | 97.5% | 268 | 89.1% | 97.4% | 347 |
| EAC**) | | | | | | | | | |
| NPV (NDBE) | 70.0% | 66.7% | 79 | 59.1% | 57.6% | 268 | 62.5% | 60.2% | 347 |

*Not an independent category, but the sum of the above HGD and EAC groups.
**cases represent the sum of LGD, HGD and EAC groups above.

TABLE 7
Fraction of the Aneuploid Samples with a Particular Chromosome Arm
Alteration in Progression from NDBE to EAC for the training Set

| Train [symbol] | | | | | | | | | | | | | | | | |
|---|---|---|---|---|---|---|---|---|---|---|---|---|---|---|---|---|
| ng | | | | | | | | | | | | | | | | |
| Set | chr1p | chr1q | chr2p | chr2q | chr3p | chr3q | chr4p | chr4q | chr5p | chr5q | chr6p | chr6q | chr7p | chr7q | chr8p | chr8q |
| BE | 0.00 | 0.00 | 0.00 | 0.00 | 0.40 | 0.40 | 0.00 | 0.00 | 0.00 | 0.20 | 0.00 | 0.00 | 0.00 | 0.00 | 0.80 | 1.00 |
| gains | | | | | | | | | | | | | | | | |
| (n = 5) | | | | | | | | | | | | | | | | |
| HGD | 0.18 | 0.27 | 0.18 | 0.36 | 0.18 | 0.36 | 0.09 | 0.09 | 0.00 | 0.09 | 0.09 | 0.09 | 0.45 | 0.18 | 0.45 | 0.64 |
| gains | | | | | | | | | | | | | | | | |
| (n = 1 [symbol] ) | | | | | | | | | | | | | | | | |
| EAC | 0.18 | 0.61 | 0.39 | 0.50 | 0.14 | 0.50 | 0.07 | 0.00 | 0.57 | 0.00 | 0.18 | 0.21 | 0.71 | 0.57 | 0.36 | 0.82 |
| gains | | | | | | | | | | | | | | | | |
| (n = 2 [symbol] ) | | | | | | | | | | | | | | | | |
| Fract [symbol] | 0.18 | 0.61 | 0.39 | 0.50 | −0.26 | 0.10 | 0.07 | 0.00 | 0.57 | −0.20 | 0.18 | 0.21 | 0.71 | 0.57 | −0.44 | −0.18 |
| on | | | | | | | | | | | | | | | | |
| EAC | | | | | | | | | | | | | | | | |
| minu [symbol] | | | | | | | | | | | | | | | | |
| Fract [symbol] | | | | | | | | | | | | | | | | |
| on | | | | | | | | | | | | | | | | |
| BE | | | | | | | | | | | | | | | | |
| BE | 0.20 | 0.00 | 0.20 | 0.00 | 0.20 | 0.20 | 0.00 | 0.20 | 0.00 | 0.00 | 0.40 | 0.20 | 0.00 | 0.00 | 0.00 | 0.00 |
| losse [symbol] | | | | | | | | | | | | | | | | |
| (n = 5) | | | | | | | | | | | | | | | | |
| HGD | 0.09 | 0.00 | 0.18 | 0.18 | 0.09 | 0.09 | 0.45 | 0.36 | 0.36 | 0.36 | 0.18 | 0.27 | 0.00 | 0.09 | 0.00 | 0.00 |
| losse [symbol] | | | | | | | | | | | | | | | | |
| (n = 1 [symbol] ) | | | | | | | | | | | | | | | | |
| EAC | 0.25 | 0.00 | 0.11 | 0.11 | 0.32 | 0.04 | 0.43 | 0.57 | 0.07 | 0.75 | 0.11 | 0.18 | 0.04 | 0.11 | 0.21 | 0.04 |
| losse [symbol] | | | | | | | | | | | | | | | | |
| (n = 2 [symbol] ) | | | | | | | | | | | | | | | | |
| Fract [symbol] | 0.05 | 0.00 | −0.09 | 0.11 | 0.12 | −0.16 | 0.43 | 0.37 | 0.07 | 0.75 | −0.29 | −0.02 | 0.04 | 0.11 | 0.21 | 0.04 |
| on | | | | | | | | | | | | | | | | |
| EAC | | | | | | | | | | | | | | | | |
| minu [symbol] | | | | | | | | | | | | | | | | |
| Fract [symbol] | | | | | | | | | | | | | | | | |
| on | | | | | | | | | | | | | | | | |
| BE | | | | | | | | | | | | | | | | |

| Train [symbol] | | | | | | | | | | | | |
|---|---|---|---|---|---|---|---|---|---|---|---|---|
| ng | | | | | | | | | | | | |
| Set | chr9p | chr9q | chr10p | chr10q | chr11p | chr11q | chr12p | chr12q | chr13q | chr14q | cqr15q | chr16p |
| BE | 0.00 | 0.20 | 0.20 | 0.20 | 0.00 | 0.00 | 0.20 | 0.20 | 0.20 | 0.00 | 0.00 | 0.00 |
| gains | | | | | | | | | | | | |
| (n = 5) | | | | | | | | | | | | |
| HGD | 0.00 | 0.45 | 0.09 | 0.27 | 0.00 | 0.18 | 0.18 | 0.18 | 0.18 | 0.36 | 0.45 | 0.18 |
| gains | | | | | | | | | | | | |
| (n = 1 [symbol] ) | | | | | | | | | | | | |
| EAC | 0.11 | 0.21 | 0.29 | 0.29 | 0.21 | 0.18 | 0.46 | 0.29 | 0.43 | 0.14 | 0.29 | 0.32 |
| gains | | | | | | | | | | | | |
| (n = 2 [symbol] ) | | | | | | | | | | | | |
| Fract [symbol] | 0.11 | 0.01 | 0.09 | 0.09 | 0.21 | 0.18 | 0.26 | 0.09 | 0.23 | 0.14 | 0.29 | 0.32 |
| on | | | | | | | | | | | | |
| EAC | | | | | | | | | | | | |
| minu [symbol] | | | | | | | | | | | | |
| Fract [symbol] | | | | | | | | | | | | |
| on | | | | | | | | | | | | |
| BE | | | | | | | | | | | | |
| BE | 0.20 | 0.00 | 0.00 | 0.00 | 0.00 | 0.20 | 0.00 | 0.00 | 0.00 | 0.20 | 0.40 | 0.00 |
| losse [symbol] | | | | | | | | | | | | |
| (n = 5) | | | | | | | | | | | | |
| HGD | 0.55 | 0.09 | 0.18 | 0.09 | 0.36 | 0.09 | 0.18 | 0.36 | 0.45 | 0.00 | 0.00 | 0.00 |
| losse [symbol] | | | | | | | | | | | | |
| (n = 1 [symbol] ) | | | | | | | | | | | | |
| EAC | 0.57 | 0.14 | 0.11 | 0.36 | 0.21 | 0.18 | 0.00 | 0.25 | 0.11 | 0.36 | 0.39 | 0.29 |
| losse [symbol] | | | | | | | | | | | | |
| (n = 2 [symbol] ) | | | | | | | | | | | | |
| Fract [symbol] | 0.37 | 0.14 | 0.11 | 0.36 | 0.21 | −0.02 | 0.00 | 0.25 | 0.11 | 0.16 | −0.01 | 0.29 |
| on | | | | | | | | | | | | |
| EAC | | | | | | | | | | | | |
| minu [symbol] | | | | | | | | | | | | |
| Fract [symbol] | | | | | | | | | | | | |
| on | | | | | | | | | | | | |
| BE | | | | | | | | | | | | |

| Train [symbol] | | | | | | | | | | | |
|---|---|---|---|---|---|---|---|---|---|---|---|
| ng | | | | | | | | | | | |
| Set | chr16q | chr17p | chr17q | chr18p | chr18q | chr19p | chr19q | chr20p | chr20q | chr21q | chr22q |
| BE | 0.00 | 0.00 | 0.00 | 0.00 | 0.00 | 0.00 | 0.00 | 0.00 | 0.20 | 0.00 | 0.00 |
| gains | | | | | | | | | | | |
| (n = 5) | | | | | | | | | | | |
| HGD | 0.09 | 0.00 | 0.45 | 0.00 | 0.09 | 0.00 | 0.18 | 0.00 | 0.27 | 0.00 | 0.00 |
| gains | | | | | | | | | | | |
| (n = 1 [symbol] ) | | | | | | | | | | | |
| EAC | 0.07 | 0.00 | 0.39 | 0.21 | 0.14 | 0.00 | 0.39 | 0.29 | 0.82 | 0.04 | 0.11 |
| gains | | | | | | | | | | | |
| (n = 2 [symbol] ) | | | | | | | | | | | |
| Fract [symbol] | 0.07 | 0.00 | 0.39 | 0.21 | 0.14 | 0.00 | 0.39 | 0.29 | 0.62 | 0.04 | 0.11 |
| on | | | | | | | | | | | |
| EAC | | | | | | | | | | | |
| minu [symbol] | | | | | | | | | | | |
| Fract [symbol] | | | | | | | | | | | |
| on | | | | | | | | | | | |
| BE | | | | | | | | | | | |
| BE | 0.20 | 0.20 | 0.20 | 0.00 | 0.20 | 0.40 | 0.40 | 0.00 | 0.00 | 0.40 | 0.20 |
| losse [symbol] | | | | | | | | | | | |
| (n = 5) | | | | | | | | | | | |
| HGD | 0.00 | 0.82 | 0.09 | 0.27 | 0.36 | 0.36 | 0.18 | 0.18 | 0.00 | 0.45 | 0.45 |
| losse [symbol] | | | | | | | | | | | |
| (n = 1 [symbol] ) | | | | | | | | | | | |
| EAC | 0.46 | 0.68 | 0.11 | 0.11 | 0.54 | 0.57 | 0.18 | 0.11 | 0.00 | 0.71 | 0.50 |
| losse [symbol] | | | | | | | | | | | |
| (n = 2 [symbol] ) | | | | | | | | | | | |
| Fract [symbol] | 0.26 | 0.48 | −0.09 | 0.11 | 0.34 | 0.17 | −0.22 | 0.11 | 0.00 | 0.31 | 0.30 |
| on | | | | | | | | | | | |
| EAC | | | | | | | | | | | |
| minu [symbol] | | | | | | | | | | | |
| Fract [symbol] | | | | | | | | | | | |
| on | | | | | | | | | | | |
| BE | | | | | | | | | | | |

[symbol]

indicates data missing or illegible when filed

TABLE 8
Sensitivity and Specificity of the entire validation
set, vs the subset of centrally-reviewed sample set

| | Entire Validation | | | Centrally reviewed Validation | | |
|---|---|---|---|---|---|---|
| | Set BE-EAC N = 184 | | | Set BE-EACN = 139 | | |
| | GAS | | | GAS | | |
| | (very- | | | (very- | | |
| | BAD + | | | BAD + | | |
| | maybe- | VERY- | # | maybe- | VERY- | # |
| | BAD) | BAD | Patients | BAD) | BAD | Patients |
| Specificity NDBE | 63.4% | 92.7% | 41 | 64.1% | 92.3% | 39 |
| Sensitivity LGD | 71.9% | 50.0% | 32 | 73.7% | 42.1% | 19 |
| Sensitivity HGD | 71.4% | 67.9% | 28 | 68.2% | 63.6% | 22 |
| Sensitivity EAC | 98.8% | 96.4% | 83 | 98.3% | 98.3% | 59 |
| Sensitivity | 91.9% | 89.2% | 111 | 90.1% | 88.9% | 81 |
| EAC + HGD | | | | | | |
| combined* | | | | | | |

*Not an independent category, but the sum of the above HGD and EAC groups.
Note:
p-value > 0.05 for all comparisons between the entire validation set and the subset by Fisher's exact two-sided test.

TABLE 9
Fraction of the Aneuploid Samples with a Particular Chromosome
Arm Alteration in Progression from NDBE to EAC for all

| | chr1p | chr1q | chr2p | chr2q | chr3p | chr3q | chr4p | chr4q |
|---|---|---|---|---|---|---|---|---|
| BE gains | 0.00 | 0.00 | 0.10 | 0.10 | 0.25 | 0.30 | 0.00 | 0.00 |
| LGD gains | 0.09 | 0.09 | 0.00 | 0.09 | 0.26 | 0.26 | 0.00 | 0.13 |
| HGD gains | 0.16 | 0.35 | 0.23 | 0.32 | 0.32 | 0.48 | 0.10 | 0.03 |
| EAC gains | 0.18 | 0.55 | 0.40 | 0.54 | 0.18 | 0.50 | 0.03 | 0.03 |
| Fraction EAC minus | 0.18 | 0.55 | 0.30 | 0.44 | −0.07 | 0.20 | 0.03 | 0.03 |
| Fraction BE | | | | | | | | |
| pvalue EAC vs BE | 1.79E−109 | 0 | 5.68E−23 | 8.80E−52 | 0.1497166 | 2.34E−06 | 0.035142 | 0.035142 |
| (Binomial | | | | | | | | |
| Proportions Test) | | | | | | | | |
| BE losses | 0.10 | 0.05 | 0.10 | 0.10 | 0.05 | 0.05 | 0.05 | 0.15 |
| LGD losses | 0.13 | 0.17 | 0.09 | 0.00 | 0.00 | 0.00 | 0.09 | 0.17 |
| HGD losses | 0.13 | 0.03 | 0.16 | 0.16 | 0.13 | 0.03 | 0.48 | 0.45 |
| EAC losses | 0.27 | 0.08 | 0.07 | 0.08 | 0.32 | 0.11 | 0.51 | 0.59 |
| Fraction EAC minus | 0.17 | 0.03 | −0.03 | −0.02 | 0.27 | 0.06 | 0.46 | 0.44 |
| Fraction BE | | | | | | | | |
| pvalue EAC vs BE | 1.46E−12 | 0.498829 | 0.4988293 | 0.7141726 | 6.46E−41 | 0.002457 | 8.31E−125 | 5.70E−48 |
| (Binomial | | | | | | | | |
| Proportions Test) | | | | | | | | |

TABLE 10
Demographics and Ploidy Results of Patients with Duplicate Specimens

| Patient | | | ID | ID | Age | | | Former or | Ploidy |
|---|---|---|---|---|---|---|---|---|---|
| Study | Patient | | Brushing | Brushing | [>90 = | | | Current | Brushing #1 |
| Number | custom IDs | Diagnosis | # 1 | #2 | 90] | Race | Gender | Smoker | Result |
| 6 | AA-0168-01-0001 | Normal | 22401 | 24033 | 53 | White | Female | Unknown | Euploid |
| 162 | AA-0686-01-0001 | LGD | 22259 | 23939 | 62 | White | Male | Yes | Euploid |
| 167 | AA-1633-01-0001 | LGD | 22332 | 23983 | 90 | White | Male | Yes | Aneuploid |
| 202 | AA-1739-01-0001 | HGD | 22351 | 24001 | 65 | White | Male | Yes | Euploid |
| 203 | AA-1746-01-0001 | HGD | 22353 | 24003 | 62 | White | Female | Yes | Aneuploid |
| 294 | AA-1747-01-0001 | EAC | 22374 | 23880 | 52 | White | Male | Yes | Aneuploid |
| 295 | AA-1748-01-0001 | EAC | 22375 | 23881 | 67 | White | Female | Yes | Aneuploid |
| 204 | AA-1749-01-0001 | HGD | 22354 | 24004 | 81 | White | Female | Yes | Aneuploid |
| 205 | AA-1755-01-0001 | HGD | 22355 | 24005 | 68 | Black | Male | Yes | Aneuploid |
| 206 | AA-1757-01-0001 | HGD | 22356 | 24006 | 61 | White | Female | No | Aneuploid |
| 171 | AA-1765-01-0001 | LGD | 22336 | 23985 | 65 | White | Male | Yes | Aneuploid |
| 45 | AA-1778-01-0001 | Normal | 22402 | 24037 | 28 | White | Male | Yes | Euploid |
| 46 | AA-1779-01-0001 | Normal | 22403 | 24038 | 47 | Black | Female | No | Euploid |
| 48 | AA-1781-01-0001 | Normal | 22405 | 24040 | 85 | White | Male | No | Euploid |
| 49 | AA-1782-01-0001 | Normal | 22406 | 24041 | 76 | White | Female | Yes | Euploid |
| 50 | AA-1783-01-0001 | Normal | 22407 | 24042 | 61 | White | Female | No | Euploid |
| 299 | AA-1784-01-0001 | EAC | 22376 | 23921 | 54 | White | Male | Yes | Aneuploid |
| 300 | AA-1785-01-0001 | EAC | 22377 | 23922 | 76 | White | Male | Yes | Aneuploid |
| 301 | AA-1786-01-0001 | EAC | 22378 | 23884 | 65 | White | Male | Yes | Aneuploid |
| 172 | AA-1790-01-0001 | LGD | 22307 | 23956 | 57 | White | Male | No | Aneuploid |
| 54 | AA-1791-01-0001 | Normal | 22422 | 24044 | 69 | White | Male | No | Euploid |
| 56 | AA-1795-01-0001 | Normal | 22425 | 24046 | 64 | White | Female | No | Euploid |

One case of discordant brushings highlighted. For this one patient with discordant brushings one brush was Euploid and not-BAD, while the du

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A method of detecting Barrett's esophagus with low grade dysplasia, or Barrett's esophagus with high grade dysplasia, or adenocarcinoma of the esophagus, the method comprising applying a Repetitive Element Aneuploidy Sequencing System (RealSeqS) methodology to a biological sample from the esophagus of the subject to detect Barrett's esophagus with low grade dysplasia, or Barrett's esophagus with high grade dysplasia, or adenocarcinoma of the esophagus.

2. (canceled)

3. (canceled)

4. The method of claim 1, wherein the application of RealSeqS methodology comprises determining a global aneuploidy score (GAS).

5. The method of claim 4, wherein the RealSeqS methodology includes (i) amplifying unique loci of genomic nucleic acid of the sample, (ii) matching the unique loci to a control, (iii) calculating the statistical gains or losses for each of non-acrocentric chromosome arms, (iv) integrating the chromosome arms into a global aneuploidy score (GAS) using machine learning, and (v) quantifying chromosome arm levels and querying focal changes of interest.

6. A method of detecting Barrett's esophagus with low grade dysplasia, or Barrett's esophagus with high grade dysplasia, or adenocarcinoma of the esophagus, the method comprising applying a Repetitive Element Aneuploidy Sequencing System (RealSeqS) methodology to a biological sample from the esophagus of a subject to determine the global aneuploidy score (GAS) and/or to identify copy number alterations in a panel of chromosome alterations, the chromosomal alterations including chromosome gains of any of chromosome regions 8q24, 1 q, 7p, 20q, 2q, 13q, 5p, 12p and/or losses of any of chromosome regions 5q, 17p, 4p, 4q, 9p, 18q, 16q, 21q, 22q, 10p.

7. (canceled)

8. (canceled)

9. The method of claim 6, wherein the global aneuploidy score indicative of presence of dysplasia or cancer, or increased risk of progression to low grade dysplasia, or high grade dysplasia, or cancer, is >0.1, >0.2, >0.3, >0.4, or >0.6, or >0.8, or >0.9, or >0.907.

10. The method of claim 6, wherein copy number alterations are determined in a panel of chromosome alterations comprising: chromosome gains of any of chromosome regions 8q24, 1q, 20q, 12p and/or losses of any of chromosome regions 17p, 9p, 10p.

11. The method of claim 6, wherein the copy number alterations are determined in a panel of chromosomes selected from 1 q, 2q, 4q, 5q, 7p, 7q, 9p, 12p, 17p, or 20q.

12. The method of claim 6, wherein the global aneuploidy score indicative of presence of dysplasia or cancer, or increased risk of progression to low grade dysplasia, or high grade dysplasia, or cancer, is >0.4, or >0.6, or >0.8, or >0.9, or >0.907 and when copy number alterations are determined in a panel of chromosome alterations, the chromosome alterations including chromosome gains of any of chromosome regions 8q24, 1q, 20q, 12p and/or losses of any of chromosome regions 17p, 9p, 10p.

13. The method of claim 12, wherein the global aneuploidy score indicative of presence of dysplasia or cancer, or increased risk of progression to low grade dysplasia, or high grade dysplasia, or cancer, is >0.6, and when copy number alterations are determined in a panel of chromosome alterations comprising: chromosome gains of any of chromosome regions 8q24, 1q, 20q, 12p and/or losses of any of chromosome regions 17p, 9p, 10p.

14. The method of claim 13, wherein the global aneuploidy score indicative of presence of dysplasia or cancer, or increased risk of progression to low grade dysplasia, or high grade dysplasia, or cancer, is >0.6, and when copy number alterations are determined in a panel of chromosome alterations comprising: chromosome gains of any of chromosome regions 8q24, 1q, 20q, 12p and/or losses of any of chromosome regions 17p, 9p.

15. The method of claim 6, wherein chromosomal gains are identified by any of values of Z_{w} (also denoted as Z) of >2.0, >2.1>, >2.2> <2.3, >2.4>, >2.5, >2.6, >2.7, >2.8, >2.9, >3.0, or by a value exceeding a cutoff between 2.0 to 3.0, and when chromosomal losses are identified by any of values of Z_{w} (also denoted as Z) of <−2.0, <−2.1, <−2.2, <−2.3, <−2.4, <−2.5, <−2.6, <−2.7, <−2.8, <−2.9, <−3.0, or by a value lower than a cutoff between −2.0 to −3.0.

16. The method of claim 1, wherein the biological sample of the esophagus is a brushing sample.

17. The method of claim 16, wherein the brushing sample is obtained by a cytology brush.

18. The method of claim 16, wherein the brushing sample is obtained by a balloon sampling device.

19. The method of claim 15, wherein the brushing sample is frozen.

20. The method of claim 1, wherein if the subject is determined to have an BE with LGD, BE with HGD, or EAC, then the method further comprises administering to the subject cryotherapy, photodynamic therapy (PDT); radiofrequency ablation (RFA); laser ablation; argon plasma coagulation (APC); electrocoagulation (electrofulguration); esophageal stent, surgery, and/or a therapeutic agent.

21. The method of claim 20, wherein the therapeutic agent is a proton pump inhibitor, a Histamine H2 receptor blocking agents, an anti-reflux medication, a drug that moves food thru the gastrointestinal tract more quickly, carboplatin and paclitaxel (Taxol), which is optionally administered in combination with radiation; cisplatin and 5-fluorouracil (5-FU), which optionally administered in combination with radiation; ECF: epirubicine (Ellence), cisplatin, and 5-FU; DCF: docetaxel (Taxotere), cisplatin, and 5-FU; Cisplatin with capecitabine (Xeloda); oxaliplatin and either 5-FU or capecitabine; doxorubicin (Adriamycin), bleomycin, mitomycin, methotrexate, vinorelbine (Navelbine), topotecan, and irinotecan (Camptosar), trastuzumab, and/or ramucirumab.

22. The method of claim 20, wherein the surgery is endoscopic mucosal resection (EMR), esophagectomy, and/or anti-reflux surgery.

23-30. (canceled)
